(19) United States
(12) Patent Application Publication
(10) Pub. No.: US 2002/0049391 A1
(43) Pub. Date: Apr. 25, 2002

(54) Method and apparatus for collecting blood

(76) Inventors: Thomas C. Kuracina, Ventura, CA (US); Randall E. Ohnemus, Ventora, CA (US)

Correspondence Address: STETINA BRUNDA GARRED & BRUCKER, 75 ENTERPRISE, SUITE 250, ALISO VIEJO, CA, 92656, US

(21) Appl. No.: 09/917,978

(22) Filed: Jul. 30, 2001

Related U.S. Application Data
(63) Continuation-in-part of application No. 09/610,027, filed on Jul. 3, 2000.
(63) Continuation-in-part of application No. 09/057,335, filed on Apr. 8, 1998, now abandoned.
(60) Provisional application No. 60/042,978, filed on Apr. 8, 1997.
(60) Provisional application No. 60/055,517, filed on Aug. 13, 1997.
(60) Provisional application No. 60/062,292, filed on Oct. 17, 1997.

(51) Int. Cl. A61B 5/00

(52) U.S. Cl. 600/576; 600/579

(57) ABSTRACT

A two-piece sealing plug for facilitating controlled regulation of a specimen flow to mitigate occurrence of hemolysis. The sealing plug has a pierceable section adapted to receive a needle with a lumen extending therein. In addition, the sealing plug has a flow diverting section conformed to slide the needle therethrough, wherein its first end is attachable to the pierceable section and its second end forms a plurality of alternate configurations to facilitate the controlled regulation of the specimen flow extruding from the lumen.

### CROSS-REFERENCE TO RELATED APPLICATIONS

This invention disclosure is related to and claims the benefit of filing dates of the following U.S. Provisional Patent Applications: (1) Ser. No. 60/042,978, entitled METHOD AND APPARATUS FOR REGULATING SPECIMEN FLOW TO A COLLECTION CONTAINER, filed Apr. 8, 1997; (2) Ser. No. 60/055,517, entitled IMPROVED METHOD AND APPARATUS FOR COLLECTING BLOOD, filed Aug. 13, 1997; (3) Ser. No. 60/062,292, entitled IMPROVED METHOD AND APPARATUS FOR COLLECTING BLOOD, filed Oct. 17, 1997; (4) U.S. Non Provisional patent application Ser. No. 09/057,335, entitled METHOD AND APPARATUS FOR COLLECTING BLOOD, filed Apr. 8, 1998; and (5) U.S. Non Provisional patent application Ser. No. 09/610,027, entitled METHOD AND APPARATUS FOR COLLECTING BLOOD, filed Jul. 3, 2000.

### STATEMENT RE: FEDERALLY SPONSORED RESEARCH/DEVELOPMENT

(Not Applicable)

### BACKGROUND OF THE INVENTION

The present invention relates generally to a method and apparatus for the collection of blood specimens, and more particularly to a blood collection apparatus which diverts, channels, regulates, diffuses or controls fluid or specimen flow to a collection container to reduce the occurrence of hemolysis, reduces the probability of collapsing the blood vessel during the specimen drawing procedure, minimizes container breakage during manufacture, use and testing, improves vacuum retention in a sealed container, allows a specimen to be placed on a slide directly from a closed collection container without using a needle, or by a blunt tipped needle, and an improved blood collection needle with a manually activated needle guard.

The collection and analysis of blood is one of the most commonly used procedures in the diagnosis of many illnesses and diseases. Blood, the essential element to human, as well as animal and marine existence, consists primarily of red blood cells, which usually range from 6-8μ (microns) in size; white blood cells, normally being from 10-14μ in size, but sometimes reaching 19-20μ in size; with additional proteins and antibodies.

The dynamics involved in collecting blood involve a number of variables and the present invention addresses these both individually and in combination, allowing a more accurate, viable sample to be safely obtained.

Blood is made up of particulate cell forms suspended in a fluid medium called plasma. The blood is contained within a closed system of pumps, passageways, chambers and valves which make up the circulatory system. Blood consists mostly of red cells and plasma, 45% and 55% respectively by volume, with the gaseous carrying red cells being suspended in the fluid plasma. The blood is pumped throughout the circulatory system by the heart, and kept in a fluid suspension medium in the blood vessels. Blood cells are actually tiny, delicate living cells which must be maintained in a chemically balanced fluid environment in order to survive and properly function.

Potassium, one of a number of inorganic substances needed to maintain a healthy metabolism, is the major cation of the intracellular fluid in red cells. The average cellular concentration of potassium in red cells is 105 mmol/l, or approximately 23 times greater than that of the average serum potassium level. Additionally, the permeability of cell membranes for potassium is extremely low, so rapid shifts of potassium in or out of cell membranes by diffusion are unlikely. Hemolyzed blood results in elevated serum potassium levels because the intracellular potassium is released from the ruptured red cells into the serum. Thus, hemolysis invalidates measured serum potassium levels.

FIG. 1 illustrates a cross-sectional and full view of a prior art blood collection device 10 that is used to withdraw a blood, bodily fluid or gas specimen from a patient. The prior art blood collection device has three primary components. These include: (1) a hollow container or vacuum tube 140, sealed by a puncturable diaphragm or stopper 141, for obtaining a fluid or gaseous sample; (2) a separate holder 130 which accepts and temporarily holds the evacuated container; and (3) a hollow bore hypodermic needle 121 having a lumen 124 therethrough and a sharpened distal tip distal 120 and sharpened proximal tip 123.

Needle 121 is attached to holder 130 with the proximal sharpened tip 123 residing in the interior of holder 130 and sharpened tip 120 extending away from holder 130. Proximal end of needle 121 typically includes a piercable resilient cover 125. Container 140, having an open end and closed end, and internal chamber 143 with removable sealing plug 141 placed in the open end of tube 140. Blood is drawn from a patient by first inserting the sharpened distal tip 120 of needle 121 into the blood vessel of the patient. Cover 125 inhibits the flow of blood from the proximal end of needle 121. Vacuum tube 140 is then positioned within hollow body 135 of holder 130 and slid forward in holder 130 (indicated by arrow M) allowing proximal end 123 of needle 121 to puncture sealing plug 141 of vacuum tube 140. The specimen fills the collection container 140 and then is removed from the needle holder 130.

The pressure difference between the patient's blood flowing in the blood vessel and the negative pressure in the vacuum chamber causes blood to be rapidly drawn into internal chamber 143 of vacuum tube 140.

FIG. 28 is a cross sectional view of a prior art needle 121 having a jagged inner wall 111.

FIG. 42 is a prior art collection container 140 having a chamber 143 sealed by a sealing plug or puncturable diaphragm 141.

The present invention addresses each aspect associated with the collection of venous or arterial blood and improves on each individual component: the needle; the needle holder; the collection container; the sealing plug or diaphragm; the sealing plug shield; and a collection adapter.

The present invention comprises a number of embodiments related to blood collection including a simple, one-piece flow diverting sealing plug, shown in FIGS. 3-10, whereby the specimen is “cushioned” in a fluid medium as it is collected through a hollow bore needle and fills collection container, rather than subjecting the blood to high impact and shear forces inherent in standard blood collecting equipment and procedures.

Another one piece sealing plug, described in FIGS. 11-15 and 30, impedes the specimen flow prior to entering collection container; a two piece sealing plug and flow controlling portion, are disclosed in FIGS. 16-19, where the specimen first enters an intermediate or inner chamber(s) prior to entering a collection container; an adjustable flow rate sealing plug having a diverter shown in FIGS. 24-27, allowing the user to easily start, stop or vary the specimen flow rate during the collection procedure.

A one piece sealing plug, described in FIGS. 20-23, discloses a reed valve means to control specimen flow through the sealing plug. Another one-piece sealing plug, described in FIG. 32, discloses a permeable or pre-pierced membrane to regulate or divert specimen flow through the sealing plug. Another one piece, multi-chambered sealing plug, shown in FIG. 33, comprises a chamber and recess or chamber to divert the specimen flow through the sealing plug.

The needle is addressed in FIG. 29, having a friction reducing coated inner wall, or the needle itself can be manufactured to smooth the jagged, rough inner surface produced by the current manufacturing processes when drawing metal tubing to size. These improvements are designed to reduce the high shear forces placed on the blood as it is sucked through the needle lumen. An increasing diameter needle, shown in FIG. 31, can be used with either the conventional sealing plug or any of the other sealing plugs described herein.

FIG. 30 describes a penetration-related adjustable flow blood collection system where the specimen flow is controlled by rotational movement of a container relative to needle holder.

A two component flow diverting sealing plug and shield, which isolates blood or bodily fluids from the phlebotomist or healthcare worker during blood collection procedures, is shown in FIGS. 34, 35, 40 and 41, with sealing plug with a chamber and covering shield having the chamber manufactured in a pre-determined position whereby needle tip penetrates only into the chamber, and not directly into the internal chamber of the collection container.

Another two component sealing plug and diverter, shown in FIGS. 47 and 48, has a means to divert the specimen radially towards the perimeter of a collection container as it exits needle lumen within the chamber, sealing plug and diverter allow specimen to enter the internal chamber of a collection container at the lower extremity, regardless of how the collection container is positioned in needle holder, where specimen can fill into itself, thus cushioning entry into collection container. Separate components of these sealing plugs are shown in FIGS. 49-52, although a multitude of configurations may also achieve the same, or similar, result.

The two component sealing plug and diverter, shown in FIGS. 47 and 48 could also be manufactured as a single component to achieve a similar desired result.

Another two piece sealing plug and diverter, shown in FIGS. 38 and 39, discloses a lower cost, reduced mass sealing plug and a diverting means to fill the container with specimen beginning at the sealing cap and filling to the opposite end of the container. The blood collection needle punctures the sealing plug concentrically and the specimen flows through the concentrically positioned diverter. A longer needle typically used to draw collected specimen from the collection container may by-pass the diverter be being inserted away from the center of the sealing plug. The sealing plug does not have to be removed to withdraw a specimen from the collection container.

A sealing plug with a reducing chamber is shown in FIG. 36 which regulates and diverts the specimen flow from a needle to a container. The reducing section also slows the specimen flow to reduce vein collapse probability during the collection process. A sealing plug, shown in FIG. 37, has a filtering means within, in or adjacent to a sealing plug or container; an adjustable depth penetration sealing plug with shield, shown in FIGS. 40 and 41, allow the user to easily start, stop or vary the specimen flow rate with a control means limiting the longitudinal movement of collection container towards needle, yet an unrestricted withdrawal of container from needle holder. The shield is included, but not necessary to practice this embodiment of the invention.

Container, shown in FIGS. 43 and 44, has a coating or film about the outside glass or plastic surface of container, reducing the probability of container breakage in the event the container is dropped or crushed during manufacturing, storage or use; container, shown in FIG. 45, has a coating, film or label about the intersection of the sealing plug and container, reducing the probability of vacuum leakage from within chamber.

Another two component sealing plug and sensor, shown in FIG. 46, has a sensor or probe which allows analysis of specimen without removal from collection container.

An automatically shielding sealing plug and shield are described in FIGS. 53 and 54 where sealing plug is slidable relative to a shield. The container has a greater gripping force on the shield whereby axial movement of the sealing plug occurs first and projection of the shield engages the sealing plug during removal with the sliding shield closing the port at an intersection.

A flow indicator or viewing section is shown in FIG. 55 allowing user to observe specimen flow in the chamber of the sealing plug. A collection container having round ends for inserting either end into a centrifuge is shown in FIG. 56. A coupling device is shown in FIG. 57 which allows a smaller pediatric needle holder, shown in FIG. 59, to be used with a standard, or larger collection container. The use of a smaller diameter device allows a shallow angle to be used for easier access of a blood vessel during blood collection procedures.

A coupling device having a flow regulating or diverting plug is shown in FIG. 58, coupling device or extension allows a smaller pediatric needle holder to be used with a larger collection container. A new collection container having two pierceable ends which can be used with both small and large needle holders is shown in FIG. 59. Larger end of collection container having a rounded contour for insertion into a centrifuge. Both sealing plugs comprise chambers, for diverting specimen flow prior to entry into collection container.

A one piece sealing plug with venting means is shown in FIGS. 61-63 to equalize the internal pressure within a collection container with the ambient atmospheric pressure prior to full removal of the sealing plug from a collection container.

A sealing plug with a chamber having external access is shown in FIGS. 64 and 65. A removable cover or seal allows the specimen deposited in the chamber to be placed on a slide without using a needle to access the specimen in the container.

A sealing plug with a chamber having external access and a movable shield with an aperture is shown in FIGS. 66-69. The movable shield allows the specimen deposited in the chamber to be placed on a slide without using a needle to access the specimen in the container.

The present invention also comprises a simple, two-piece flow diffusing sealing plug, shown in FIGS. 70, 71, and 71A, whereby the specimen is “diffused” in a porous medium as it exits the hollow bore needle prior to filling a collection container.

FIG. 71B shows a simple flow regulating means within a needle lumen, allowing a specimen to be collected with a conventional, evacuated blood collection container yet reducing both hemolysis and vein collapse probability.

Another simple, two-piece flow diverting sealing plug, is shown in FIGS. 72 and 73, whereby the specimen is diverted through an aperture during the collection process. An expandable material creating the aperture is activated by a contact with a liquid and swells to close the aperture within minutes of becoming wetted.

A two-piece sealing plug which adds a single diverting component to an existing prior art sealing plug or the like is shown in FIG. 74. The diverting component may also include a supporting means to keep the sealing plug wall from collapsing or moving when positioned in the tube, thus improving vacuum retention inside the tube by maintaining a proper seal between a sealing plug and tube. A compressive, radial force is exerted on the elastomeric sealing plug wall by the inner support or diverting component. The distal diverter wall section between the chamber created within the sealing plug and collection container is positioned far enough away from the blood collection needle so the needle does not puncture the wall section when a specimen is collected. The distal diverter wall section may be puncturable by a longer needle which safely draws out the collected specimen from the container during testing and analysis without removing the sealing plug. The diverting wall section may also include a dissolvable or separable material. The separable wall section would be sufficiently held in place during the collection process, and removed by the centrifugal forces created when the collection container is centrifuged.

Another two-piece sealing plug with a supporting means to keep the sealing plug wall from collapsing or moving when positioned in the tube is shown in FIG. 75. This invention improves vacuum retention inside the tube by maintaining a proper seal between a sealing plug and container wall.

A two-piece sealing plug is shown in FIGS. 76 and 77 included a diverting component which is activated when a pressure difference is created between the intermediate chamber of the sealing plug and the internal chamber of the collection tube. A valve opens when a specimen is drawn into the chambers, and closes when the pressure in the chambers is substantially equal.

A sealing plug with a diverting component with a channel or slot is shown in FIGS. 78 and 79. The specimen is collected through the diverter which is concentrically positioned to accept a puncturing blood collection needle. A channel is eccentrically positioned to allow a long needle to be inserted into the collected specimen without contacting any specimen which may remain in the intermediate chamber of the sealing plug and diverter after the specimen is collected.

A blood collection needle is shown in FIG. 80 with a needle guard and a positive stop to keep the needle guard adjacent to the needle hub during the blood collection procedure. A compressive force exerted on a finger pad or button selectively releases the needle guard from a retained position to a protecting position where the sharp needle tip is safely covered when the user so desires.

There are no known blood collecting devices which take the delicate physical nature of living blood cells into consideration during the collection process as thoroughly and comprehensively as the present invention does.

Standard blood collection vacuum tubes are popular for everyday blood drawing procedures, with a variety of additives such as anti-clotting agents, clotting agents, wax, reagents or the like included in the evacuated chamber to facilitate the examination of the blood specimen. Basically, all standard, single chambered vacuum tubes are designed to draw fluids or gaseous substances into the evacuated chamber at a rapid, uncontrolled rate. Single chambered vacuum tubes comprise a single negative pressure in a chamber 143, which causes a rapid suction of fluids or gaseous substances to be drawn through a small, hollow bore needle lumen 124 and into evacuated chamber 143 when sealing plug 141 is punctured by the needle. The manner in which a specimen is drawn into chamber 143 causes high forces to act on the delicate blood cells.

Since the introduction of evacuated blood collection containers, widely known as the VACUTAINER® brand blood collection system, described in FIG. 1, improvements have been limited to sharper needles, new additives for inside the collection container, tubes made of plastic resin to reduce container breakage, and vacuum retention to improve shelf life of the collection tube.

There are major limitations inherent to the use of standard, single chambered blood collection vacuum tubes. First, the unrestricted, high velocity flow rate of the specimen through the hollow bore needle into the evacuated chamber launches the specimen into an empty chamber on a collision course with the far wall of container, causing physical damage to the blood cells, or even cell membrane rupture, which is also known as hemolysis. Secondly, the unrestricted suction pressure of vacuum tube often results in the collapse of the patient's blood vessel, which then requires the use of a syringe to obtain the specimen.

The pressure difference between the existing blood pressure of the patient and the sub-atmospheric pressure in the evacuated collection container determines the velocity and flow rate of the specimen entering the container. The sub-atmospheric pressure in evacuated chamber is greatest when the sealing plug is initially punctured by the needle. The specimen is uncontrollably projected at a high velocity through the empty chamber, impacting the far wall of container.

Furthermore, standard blood collection equipment and procedures place the delicate blood specimen in a very precarious environment, creating high shear forces as the specimen is sucked through and exits the small, hollow bore needle. The shear forces may hemolyze the blood cells. The velocity and momentum of the specimen entering the vacuum container causes it to be launched across an empty chamber and into the hard, unforgiving far wall of the collection container. The flow velocity of the specimen entering the container is great enough to injure and rupture many red cells as they impact the container wall, causing hemolysis. Many of the blood cells which initially survive the impact with the far wall of the container intact may be injured due to the serious blunt force trauma imposed on the cells.

Of course, the main objective of collecting the specimen is to determine the health of the patient. This is best accomplished by keeping the blood specimen intact, and in the most viable condition possible, in other words, alive and living.

During analysis, some specimens are found to be so damaged and grossly hemolyzed during the collection process, that the laboratory issues a disclaimer and orders another blood sample taken from the patient for analysis. The re-drawing of a specimen creates an additional, unnecessary cost for the medical institution and requires the patient to be punctured with a needle a second or third time.

In the history of blood collection, few attempts have been made to prevent hemolysis from occurring during the collection process. The majority of hemolysis related prior art simply makes adjustments to the test results, essentially compensating for the damage inflicted on the specimen during the collection process. This “after the fact” procedure further complicates analysis and is at best a speculative attempt to determine the health of the specimen, and patient.

The most notable attempt to prevent hemolysis during the collection process is taught by Villa-Real in U.S. Pat. No. 4,492,634 where a sealing plug with a baffle extension simply deflects the high velocity stream of blood into the side wall of the collection tube. This deflection causes the fragile blood cells to impact the hard side wall of the tube. The apparatus of Villa-Real also generates additional turbulence within the collection tube.

An earlier valvular device is also taught by Villa-Real in U.S. Pat. No. 3,848,579, which attempts to control the flow of blood passing through the hollow bore hypodermic needle. This apparatus draws blood through a small hollow bore needle into a larger diameter chamber housing a reed valve, again reducing to a hollow bore needle and exiting into a collection tube. One problem also created with the apparatus of Villa-Real is that the opening and closing of the valve causes additional turbulence and forces to act upon the delicate living blood cells during the collection process. After passing through the valve, the specimen flow path is again reduced. Additional components are also needed to fabricate this invention, increasing the cost.

The rate at which blood is drawn from a blood vessel is determined by the volume of blood in the vessel, the pressure difference between the internal pressure of blood vessel and the sub-atmospheric pressure in the collection vacuum tube, and the internal diameter size of the needle lumen. For instance, a common 21G blood collection needle has an inside diameter of approximately 0.028″, which allows an evacuated tube to be filled in only a few seconds.

Another problem which regularly presents itself during the collection process is blood vessel collapse. The volumetric capacity of the blood vessel in certain patients is inadequate to self-replenish when blood is rapidly collected into a standard collection container. This problem is typical with young, older or anxious patients.

The inadequate replenishment of blood causes the blood vessel to collapse, interfering with the blood collection procedure and forcing the phlebotomist to use a standard, sterile syringe and hypodermic needle to collect the blood sample. The syringe permits a manually operated suction to be applied to the blood vessel, thus enabling the healthcare worker to collect the blood sample at a rate that usually is slow enough to keep the vessel from collapsing.

Routinely, when a syringe is used to collect a blood specimen in a patient whose vessels are prone to collapse, the patient's blood vessels must be punctured a number of times to collect enough blood specimen for analysis. Multiple punctures are painful to the patient and can take an extra 5 to 15 minutes to complete. The healthcare worker may also become anxious because of the inability to collect the needed specimen in an allotted time without undue discomfort to the patient. The collected blood specimen must be then transferred into an evacuated tube for storage and testing.

Conversely, another problem occurs when the vacuum within the collection container diminishes or leaks over time, reducing the shelf-life of evacuated tubes. This results from an improper seal at the sealing plug/container surface interface. The seal is discontinuous because of the uneven surface inherent to molded or compression molded elastomeric materials. Small voids in the sealing plug or stopper surface allow the seal to be breached by the ambient atmosphere and the vacuum within the container is lost over time. Areas where the seal is discontinuous is known in the industry as “gray band” regions. Elastomeric materials are known to be susceptible to temperature fluctuations and typically contract over the course of time.

Vacuum leakage was so prevalent when evacuated collection containers were first introduced that the evacuated collection tubes were packaged in sealed shipping containers which also had been evacuated. The user would open the shipping container releasing the vacuum inside the container and then use the collection tubes.

Collection containers with reduced vacuum do not provide a sufficient amount of specimen needed for analysis and are normally disposed of as medical waste. Another tube must then be used to collect an adequate amount of specimen for analysis. Despite the attempts to minimize vacuum leakage in containers through computer generated sealing plugs and the use of different materials, vacuum loss still remains a problem.

The present invention provides an improved sealing means at the sealing plug/container surface interface. The improved sealing means invention can be used with prior art sealing plugs or with the flow diverting, regulating or diffusing sealing plugs disclosed in this application.

A blood specimen is normally prepared for analysis either of two ways, depending on the nature of the test to be performed. If the serum is to be analyzed, the elements of red cells and plasma remain combined as whole blood serum. The specimen is allowed to clot and retract overnight before being decanted and analyzed.

If the plasma or red cells are to be analyzed individually, they are spun in a centrifuge to separate the red cells from the plasma. Additives are normally included in the collection container to delay clotting.

The present invention also includes a flow diverting sealing plug with a liquid activated material surrounding an aperture which is in the fluid path. The blood is collected and within minutes, the liquid activated material swells closing the aperture. The closed aperture prevents any specimen remaining in the sealing plug from combining with the specimen in the collection container after the collection process is completed.

Another test, the complete blood count (CBC) test is performed on whole blood immediately after the specimen is collected.

Blood which is collected with a syringe must be transferred to an evacuated tube prior to analysis or centrifuging. Transferring the collected specimen from a hypodermic syringe to an evacuated tube is considered the most dangerous blood transferring procedure in medicine today because the healthcare worker must move the exposed needle containing blood directly towards the hand holding the evacuated tube. One small miscalculation and the healthcare worker receives a direct inoculation of the specimen blood contained in the needle lumen.

If a needlestick occurs during the transfer of blood contained in the syringe and needle to a vacuum tube, and the blood specimen contains a bloodborne pathogen such as of Human Immunodeficiency virus (HIV), Hepatitis C (HCV) or any of the other twenty-odd bloodborne pathogens which are transmitted by blood, the healthcare worker may become infected by that pathogen. Bacterial pathogens such as Yersinia enterocolitica and Pseudomonas fluorenscens have recently been identified in collected blood, further threatening the well being of healthcare workers if a needlestick occurs and the bacteria is present in the blood.

If the healthcare worker forcibly transfers the specimen from the syringe into the vacuum tube, over pressurizing the vacuum tube may cause the sealing plug to dislodge from the vacuum tube and spray the collected blood into the workplace.

Two posters highlighting the problem of transferring the blood collected in a syringe into a vacuum tube were presented at the August 1995 Centers for Disease Control conference on preventing needlesticks. One alternative solution to the problem is taught in U.S. Pat. No. 5,439,450, where the blood is collected in a user-activated 10 cc sliding sleeve syringe. This rather large size syringe has a sliding sleeve with a diameter sufficient to insert the evacuated tube into the extended sliding sleeve thereby isolating the needle from the healthcare workers' hands during the transfer from the syringe.

The standard blood collection system may work adequately in instances where patients' blood vessels have the capacity to quickly replace the blood volume being removed and collected. However, approximately five percent of blood collections must be accomplished with a manually activated syringe. This translates into approximately 150 million blood collection specimen procedures with a syringe per year.

Essentially, blood cells are living, microscopic “liquid balloons” containing liquid in a permeable membrane, which may be injured, or even rupture, when exposed to excessive force. The membrane may also break as a result of being torn or otherwise traumatized. Hemolysis of red blood cells is one problem that may result from the over traumatization of the blood sample.

In addition to hemolysis of red blood cells, vacuum drawing may result in other abnormalities which tend to confound interpretation of data relying on visual examination of cells. The appearance of cell abnormalities or cell fragments, which may be regarded as an indication of illness, may actually comprise artifact caused in the vacuum drawing process. For instance, red blood cell abnormalities such as clumping or stacking, which may otherwise be indicative of disease, may in actuality be caused by the blood drawing process, and not the disease. A more optimal sample of blood can be obtained if the blood collection procedure can be accomplished without creating unnecessary trauma to the blood cells in the sample, the circulatory system or the patient.

FIG. 2 shows a blood specimen being introduced into the internal cavity 143 of the blood collection container 140 of FIG. 1. As illustrated, once the distal end 123 of the needle 121 pierces the collection container sealing plug 141, the blood specimen rushes into the evacuated cavity 143. Initially, the blood specimen impinges the collection container wall at a high velocity. The high velocity impact of the blood specimen against the internal walls of cavity 143 traumatizes the blood specimen causing hemolysis and other undesirable physical changes to occur within the specimen.

Even as the evacuated cavity 143 fills, the specimen continues to enter the tube 140 at a high velocity causing an inner fluid and gaseous turbulence within the cavity 143.

It is also important to note that a small amount, or smear, of blood is normally placed on a slide during the collection process. The slide specimen is used to determine the percentage of white cells per 100 blood cells, or the complete blood count (CBC) by means of a visual test. The CBC test can be conducted manually, or in an automated fashion, where a laser is directed on the specimen and the cell count is determined by the way the laser bounces off the different cells.

The smear is obtained by removing the blood collecting apparatus from the venipuncture site with an needle exposed by pressing the filled collection container against the needle holder, forcing a small amount of blood through the needle and onto a slide. Sometimes the needle used to directly access the blood vessel is disposed of and a new needle is placed on the needle holder whereby a filled collection container is pressed in the needle holder to obtain a smear for a slide.

If the needle is withdrawn from the venipuncture site covered, a new needle must be used to obtain a smear for a slide. The use of another needle to obtain a smear for a slide adds cost and exposes the healthcare worker to another sharp needle containing blood.

What is needed is an apparatus and method for collecting a specimen sample, such as blood or other bodily fluids or gases into a collection container that solves the aforementioned problems.

### BRIEF SUMMARY OF THE INVENTION

The foregoing objects have been achieved by the specimen collecting apparatus of the present invention capable of regulating, restricting, controlling, diverting or varying the flow of a specimen within or into a collection container, evacuated container or syringe.

The present invention provides an improved apparatus and method for delivering a specimen sample into a collection container.

In one embodiment, a specimen collection apparatus is provided having a flow diverting or regulating means which diverts or regulates the flow of a specimen into or within a specimen collection container.

In one embodiment, a specimen collection apparatus is provided with a needle or other fluid communication means, having an occluded or restricted lumen that regulates the flow rate of a specimen into a specimen collection container.

In another embodiment, a hypodermic needle, or other fluid/gas communication means, is provided having a lumen bore that is coated with a friction reducing material, such as silicone.

In yet another embodiment, a hypodermic needle, or other fluid/gas communication means, is provided having a lumen bore that is coated with a anti-clotting material, such as heparin.

In yet another embodiment, the vacuum tube sealing plug is provided having a flow diverting or regulating means that diverts or regulates the flow into and/or within a collection container. The sealing plug contains a least one opening, channel, cavity, area or passageway which diverts or regulates the specimen flow through the sealing plug from a needle into the collection container. The flow diverting or regulating opening, channel, cavity, area or passageway is in direct communication with the internal cavity of collection container. The flow diverting or regulating opening, channel, cavity, area or passageway can also be coated with a friction reducing material, anti-clotting material or the like used in the analysis of blood.

In yet another embodiment, the sealing plug is provided having a flow diverting or regulating means that diverts or regulates the flow through, into or within the sealing plug itself. The sealing plug is provided with at least one chamber, which can be an internal, open, intermediate or inner chamber, that diverts or regulates the specimen flow into and/or within the collection container. Blood, or other bodily fluids or gases, is introduced into the chamber of the sealing plug before being directed into the internal cavity of the collection container. The chamber can be coated with a friction reducing material, anti-clotting material or the like normally used in the analysis of blood.

The sealing plug of the present invention suspends the blood in a fluid medium as it is collected, thus reducing the probability of damaging the blood elements during the collection process.

In another embodiment, the sealing plug comprises a plurality of sections which create a specimen flow diverting or regulating means. The plurality of sections can be coated with a friction reducing material, anti-clotting material or the like used in the analysis of blood.

In still another embodiment, the sealing plug comprises a plurality of sections which create a specimen flow diverting or regulating means whereby an intermediate chamber is created by joining the sealing plug sections together. The plurality of sections can also be coated with a friction reducing material, anti-clotting material or the like normally used in the analysis of blood.

In another embodiment, the sealing plug comprises a plurality of components which create a specimen flow diverting or regulating means whereby an intermediate chamber is created by joining the sealing plug and a separate component together. The plurality of sections can also be coated with a friction reducing material, anti-clotting material or the like used in the analysis of blood.

In yet another embodiment, the vacuum tube sealing/plug is provided having an adjustable flow diverting or regulating means that diverts or regulates the flow into and/or within a collection container. The flow diverting or regulating section sealing plug is contained within the container opening.

In still yet another embodiment, the vacuum tube sealing plug is provided having an adjustable flow diverting or regulating means that diverts or regulates the flow into and/or within the sealing plug. The sealing plug contains a secondary plug or section which is denser than the sealing plug and is engaged by the needle during the blood drawing procedure. The advancing needle moves the secondary plug or section allowing a port on the sealing plug to open and the specimen to enter the collection container at a controlled rate. The secondary plug or section can be coated with a friction reducing material, anti-clotting material or the like used in the analysis of blood.

In another embodiment, a vacuum tube sealing plug, or sealing closure is provided having an adjustable flow diverting or regulating means that frictionally or threadedly engages the inner section of the collection needle holder allowing the healthcare worker to adjust the specimen flow into and/or within the collection container. The engaging components may also be coated with a friction reducing material.

In one embodiment, the sealing plug includes a flow diverting or regulating means to limit the specimen flow from the chamber of the sealing plug into the inner cavity of the collection container.

In another embodiment, the sealing plug includes a connected flow diverting or regulating means to limit the specimen flow from the sealing plug into the collection container.

In one embodiment, a specimen collection apparatus is provided having a flow diverting or regulating means that diverts or regulates the flow rate of a specimen into or within a specimen collection container.

In one embodiment, a specimen collection apparatus is provided with a removable shield over the sealing plug, which protects the healthcare worker from being exposed to the patient's blood during the collection and testing procedure and also allows easy removal of the sealing plug from the collection container.

In another embodiment, the vacuum tube sealing plug is provided having a flow diverting or regulating means that diverts or regulates the flow into and/or within a collection container. The sealing cap contains a least one opening, channel, cavity or area which regulates the specimen flow through the sealing plug from a hollow bore needle into the collection container. The flow regulating opening, channel, cavity, area or passageway can also be coated with a friction reducing material, anti-clotting material or the like used in the analysis of blood.

In yet another embodiment, the vacuum tube sealing plug is provided having a two piece flow regulating means that regulates the flow into and/or within a collection container. The flow regulating section is simply pushed into the sealing plug prior to insertion into the opening of the container.

In still yet another embodiment, the vacuum tube sealing plug is provided having an filtering means that filters the specimen as it flows into and/or within the sealing plug.

In another embodiment, a vacuum tube, sealing plug, or sealing closure is provided having an adjustable flow diverting or regulating means that frictionally or threadedly engages the inner section of the collection needle holder allowing the healthcare worker to adjust the specimen flow into and/or within the collection container. The engaging components can be coated with a friction reducing material, anti-clotting material or the like used in the analysis of blood.

In one embodiment, the sealing plug includes a flow diverting or regulating means to limit the specimen flow from the chamber of the sealing plug into the inner cavity of the collection container by means of a filter or reduced passageway.

In another embodiment, the sealing plug includes a connected flow diverting or regulating means to limit the specimen flow from the sealing plug into the collection container.

In another embodiment, the position of the needle tip in the piercable sealing plug is provided so as to limit the longitudinal movement of the needle tip only within the sealing plug chamber, and not into the internal chamber of the collection container.

In yet another embodiment, a collection container is provided having an enveloping coating or film to increase shatter resistance of the collection container.

In one embodiment, a collection container is provided having a breakable polymeric, elastomeric or the like coating or film applied to the interface of the container and sealing plug to reduce the probability of vacuum leakage from within the evacuated container. This embodiment also serves as a vacuum leak or tampering indicator. Breakage of this seal or coating will alert the healthcare worker of a possible vacuum leak or tampering.

In another embodiment, a collection container is provided having a sealing plug with a sensor which is accessible from the outside of the collection container whereby the specimen can be analyzed without removal of the sealing plug.

In another embodiment, a collection container is provided having an automatically shielding sealing plug when a sealing plug is removed from a collection container. Sliding shield covers the inner surface of a sealing plug which has been in contact with a collected specimen. The sliding shield closes sealing plug chamber and traps any remaining specimen contained within chamber from coming in contact with healthcare personnel.

In another embodiment, a collection container is provided having a flow indicating or viewing area to observe specimen flow during the collection process.

In still another embodiment, a collection container is provided having either end being semi-circular for inserting either end container into a centrifuge.

In another embodiment, a coupler or extension is disclosed allowing a larger diameter collection container to be used with a smaller diameter needle holder whereby a shallower angle is available for accessing a insertion site.

In yet embodiment, a flow regulating coupler or extension is disclosed allowing a larger diameter collection container to be used with a smaller diameter needle holder whereby a shallower angle is available for accessing a insertion site.

In one embodiment, a single collection container can be used with needle holders having different diameters.

In another embodiment, a sealing plug with venting means is provided to equalize the internal pressure within the collection container with the ambient atmospheric pressure prior to full removal of the sealing plug from the collection container.

In one embodiment, a specimen is deposited in a chamber of a sealing plug having external access, allowing a specimen to be placed on a slide without using a needle to access the specimen.

In another embodiment, a needle with a sharp tip is used to gain access to a filled collection container, with the other exposed needle end having a blunted tip to reduce needlestick probability and allow a specimen to be safely placed on a slide.

In another embodiment, a container with an improved sealing plug/container surface interface is used to increase the shelf life of the evacuated container.

In yet another embodiment, a sealing plug includes a porous material to diffuse or filter a liquid being drawn into a container.

In another embodiment, a needle lumen includes a regulating means within the lumen so the specimen flow may be regulated during the collection process using a conventional, evacuated blood collection tube.

In still another embodiment, a sealing plug with a liquid activated section allows a specimen to be collected, and prevents any specimen remaining within a sealing plug to remain there during the centrifuge process.

In another embodiment, a diverting component with at least one aperture is inserted in the hollow end of a prior art sealing plug, allowing existing sealing plug tooling and existing containers to be utilized. This embodiment reduces the overall cost of implementing the flow diverting or regulating invention. The size of the aperture can be made smaller than the inner diameter area of the needle used to puncture the sealing plug, reducing the specimen flow and the probability of vein collapse. A procedure incorporating a reduced aperture size in the diverter will take longer to complete since the flow is reduced or restricted.

The aperture size can also be equal to or greater than the inner diameter area of the needle used to puncture the sealing plug, allowing full flow through the needle during the collection process. A procedure incorporating an equal or increased aperture size in the diverter will take the same amount of time as a standard blood draw.

In one embodiment, a sealing plug with a pressure sensitive valve is activated when a pressure difference exists on either side of the valve.

In another embodiment, a sealing plug is shown with and diverter having an aperture and an adjacent channel.

In another embodiment a blood collection needle with a needle guard is shown where the needle guard can be selectively activated by the user.

Wherefore, it is an object of the invention to provide a hypodermic blood collecting apparatus and method which allows a diverted, regulated, controlled, diffused, or variable specimen flow into a collection container.

It is another object of the invention to provide a hypodermic blood collecting apparatus and method which allows a regulated, controlled, governed, gated, diverted, diffused or variable specimen flow into through, or within a diaphragm or plug and into a collection container.

It is also an object of the invention to provide a hypodermic blood collecting apparatus and method which allows a regulated, controlled, governed, gated, diverted, diffused or variable specimen flow into a collection container without adding any additional parts to the apparatus.

It is another object of the invention to provide a hypodermic specimen collecting apparatus and method which allows a regulated, controlled, governed, gated, diverted, diffused or variable specimen flow into a collection container and looks and functions in a similar manner to a standard, blood collecting hypodermic needle system.

It is yet another object of the invention to provide a hypodermic blood collecting apparatus and method which allows a regulated, controlled, governed, gated, diverted, diffused or variable specimen flow into a collection container which lends itself to automated manufacturing.

It is a further object of the invention to provide a hypodermic specimen collecting apparatus and method which allows a regulated, controlled, governed, gated, diverted, diffused or variable specimen flow into a collection container where the hypodermic specimen collecting apparatus can be positioned away from the vascular access site by means of a tube or the like.

It is still another object of the invention to provide a hypodermic specimen collecting apparatus and method which allows a regulated, controlled, governed, gated, diverted, diffused or variable specimen flow into a collection container where the sealing cap of the collection container comprises a flow regulating means.

It is an additional object of this invention to provide a hypodermic specimen collecting apparatus and method which allows a regulated, controlled, governed, gated, diverted, diffused or variable specimen flow into a collection container where a variable compressive or circumferential force is used to limit or regulate the interaction between the sealing plug and the openable port of the sealing plug.

It is another object of the invention to provide a hypodermic specimen collecting apparatus and method which allows a regulated, controlled, governed, gated, diverted, diffused or variable specimen flow into a collection container where a variable compressive force is used to limit or regulate the interaction between the needle holder and the collection container.

It is also an object of the invention to provide a low-cost hypodermic specimen collecting apparatus and method which allows the specimen to primarily remain in a fluid medium, cushioning its flow into and within a collection container.

It is another object of the invention to provide a hypodermic specimen collecting apparatus and method which allows a specimen flow into a collection container where at least one diaphragm or barrier regulates, restricts, diverts or controls the specimen flow within a collection container.

It is a yet an additional object of the invention to utilize the flow regulation means to other collection or transferring procedures, such as transferring blood from a syringe to a collection container.

It is still another object of this invention to provide a specimen collecting apparatus that reduces the probability of collapsing a blood, or other bodily, vessel of a patient during a specimen drawing procedure.

It is a further object of the invention to reduce specimen trauma during the specimen collection procedure.

It is another object of the invention to provide a specimen collecting apparatus and method which minimizes the hemolysis of a specimen collected during a blood collection procedure.

It is still another object of the invention to provide a flow reducing, then increasing means on a sealing plug which regulates the specimen flow into a collection container.

It is a still further object of the invention to provide a filtering means within, in, or adjacent to, a sealing plug which regulates, controls, governs, gates, diverts or adjusts the specimen flow into a collection container.

It is another object of the invention to provide a sensing means within a sealing plug or container which determines the status of the blood, blood elements, or blood gasses while the blood is contained within the collection container, sealing plug chamber or passageway.

It is yet another object of the invention to provide a protective coating or film on a collection container to reduce breakage and leak probability during manufacturing, storage and use.

It is still another object of the invention to provide a coating or film on a collection container seal to prevent vacuum leakage of the collection container prior to use.

It is another object of the invention to preserve the shelf life of the collected specimen in the best possible condition for as long as possible.

It is another object of the invention to keep the collected specimen cells alive and living until they are analyzed.

It is an object of the invention to provide a collection container which has a sealing plug, which when removed, automatically shields the surfaces of a sealing plug which have been in contact with a specimen in a collection container.

It is another object of the invention to provide a collection container which has a flow indicating means viewable by the user.

It is another object of the invention to provide a coupler which allows a large diameter collection container to be used with a smaller diameter needle holder.

It is yet another object of the invention to provide a flow regulating coupler which allows a large diameter collection container to be used with a smaller diameter needle holder.

It is still another object of the invention to provide a single collection container which can be used with needle holders having different diameters.

It is another object of the invention to provide a needle access angle which is as close as possible to being parallel with the plane of the insertion site.

It is a further object of the invention to provide a venting means to equalize the internal pressure within a collection container with the ambient atmospheric pressure prior to full removal of a sealing plug from a collection container.

It is another object of the invention to provide access to a small amount of specimen from a collection container without using a needle to obtain the specimen.

It is still another object of the invention to provide a hollow bore needle with one sharp tip to gain access to a filled container, with the other exposed needle end having a blunted tip to reduce needlestick probability and allow a specimen to be safely placed on a slide.

It is another object of the invention to provide a means to diffuse the liquid entering a container.

It is another object of the invention to provide a means within a needle lumen to diffuse or regulate the liquid entering a container.

It is another object of the invention to provide a blood collection needle that includes a regulating means, allowing a specimen to be safely collected with a conventional, evacuated blood collection tube without hemolyzing the red cells, or collapsing the patient's blood vessel during the collection procedure.

It is still a further object of the invention to provide an improved sealing means at the sealing at the sealing plug/container surface interface.

It is still another object of the invention to provide an improved sealing means at the sealing plug/container surface interface which may include a specimen diverting, diffusing or regulating means.

It is another object of the invention to provide a sealing plug with a pressure sensitive valve means which is activated when a pressure difference exists on either side of the valve.

It is another object of the invention to provide a sealing plug with a diverting means and an adjacent channel which allows a specimen to be collected in a normal manner where the blood collection needle punctures a sealing plug in a normal concentric manner, and a longer needle for obtaining the collected specimen for analysis punctures the sealing plug in an eccentric manner without contacting any specimen residing in the chamber created by the sealing plug and diverter.

It is a further object of the invention to provide a blood collection needle where a needle guard can be selectively activated only by a manual releasing means.

For simplicity sake, the numbered components shown herein could be interchanged throughout the drawings, providing a variety of combinations of the described invention.

Other objects and benefits of this invention will become apparent from the description which follows hereinafter when read in conjunction with the drawing figures which accompany it.

### BRIEF DESCRIPTION OF THE DRAWINGS

The present invention is illustrated by way of example and is not limited by the figures of the accompanying drawings, in which like references indicate similar elements and in which:

FIG. 1 illustrates a prior art blood collection apparatus.

FIG. 2 shows a blood specimen being introduced into the prior art blood collection apparatus of FIG. 1.

FIG. 3 illustrates a full view of a sealing plug for a blood collection container having a chamber open to the side of the sealing plug.

FIG. 4 illustrates a side view of a specimen collection needle and cut away view of a needle holder with a full view of a specimen collection tube of the present invention having a sealing plug with an intermediate chamber.

FIG. 5 illustrates the blood collection apparatus of FIG. 4 with a cross sectional side view of a sealing plug.

FIG. 6 shows the apparatus of FIG. 5 with a specimen being introduced into the collection tube.

FIG. 7 illustrates a cross sectional side view of a sealing plug with one chamber.

FIG. 8 illustrates a cross sectional view of the sealing plug of FIG. 7 inserted in the open end of a container wherein the chamber has at least one aperture or port.

FIG. 9 illustrates a full bottom view of the sealing plug of FIG. 7.

FIG. 10 shows a full bottom view of one embodiment of the present invention comprising a plurality of channels.

FIG. 11 illustrates another embodiment of the present invention whereby the sealing plug regulates the specimen flow by partially impeding or blocking the specimen exiting the hollow bore of the needle lumen.

FIG. 12 shows a full bottom view of the sealing plug of FIG. 11.

FIG. 13 illustrates a cut away view of a sealing plug in another embodiment of the invention.

FIG. 14 illustrates a cut away view of the sealing plug of the present invention having a plurality of reducing channels or apertures.

FIG. 15 illustrates a cut away view of the present invention wherein the sealing plug of the specimen collection container comprises a plurality of increasing or expanding channels.

FIG. 16 illustrates one embodiment of the present invention wherein the sealing plug of the specimen collection container comprises two components.

FIG. 17 is a cross sectional view of the sealing plug shown in FIG. 16.

FIG. 18 is a cross sectional view of one embodiment of the present invention shown as molded.

FIG. 19 shows a sealing plug with a hinge ready to be inserted into the open end of a collection container.

FIG. 20 illustrates a sealing plug of the present invention having a reed valve.

FIG. 21 illustrates a sealing plug of the present invention having a recessed reed valve.

FIG. 22 illustrates the sealing plug shown in FIG. 20.

FIG. 23 illustrates the sealing plug shown in FIG. 21.

FIG. 24 illustrates another embodiment of the present invention whereby the sealing plug comprises a movable component with a closed port or aperture, and the needle holder comprises a compressive longitudinal resisting member.

FIG. 25 shows the sharpened tip of the needle engaging a movable component of the sealing plug and opening the aperture. The vacuum tube closure and needle holder provide a threaded or frictional means to adjust the specimen flow into the collection container.

FIG. 26 shows a cross sectional view of the sealing plug of the present invention with a movable component and closed aperture.

FIG. 27 shows a cross sectional view of the sealing plug of the present invention with an integrally molded movable component and closed aperture.

FIG. 28 illustrates a cross sectional view of a prior art hypodermic needle wall.

FIG. 29 illustrates a cross sectional view of a hypodermic needle wall wherein the inner wall is coated with a friction reducing material, filler or anti-clotting material.

FIG. 30 shows a vacuum tube and needle holder provided with a threaded or frictional means to adjust the specimen flow into a collection container.

FIG. 31 illustrates a cross sectional view of a needle with an expanded needle bore.

FIG. 32 illustrates a cross sectional view of a collection container with sealing plug and an adjacent chamber.

FIG. 33 shows a cross sectional view of a collection tube with a multi-chamber sealing plug.

FIG. 34 illustrates a specimen collection needle and needle holder with a specimen collection tube of the present invention having a sealing plug with an intermediate chamber, passageway and a shield engaging the sealing plug.

FIG. 35 illustrates a cross sectional side view of the assembly of FIG. 34 with the needle tip residing within the intermediate chamber.

FIG. 36 shows the apparatus of FIG. 35 with an outer sealing means and a reducing internal chamber.

FIG. 37 illustrates a partial cut away side view of a collection container having a sealing plug with one intermediate chamber and a filter means.

FIG. 38 illustrates a two piece sealing plug of the present invention wherein the intermediate chamber is created by adjoining two components together.

FIG. 39 illustrates a full bottom view of the sealing plug of FIG. 38.

FIG. 40 shows a partial cross sectional view of one embodiment of the present invention comprising an adjustable flow regulation means.

FIG. 41 illustrates an alternative side view of the apparatus shown in FIG. 40.

FIG. 42 shows a partial cut away side view of a prior art collection container.

FIG. 43 illustrates a cross sectional view of one embodiment of the present invention of a collection container with a protective coating or film on the outer surface of the container.

FIG. 44 illustrates a cross sectional view of one embodiment of the present invention of a collection container with a coating or film on the outer surface of the collection container extending over the outer surface of the interface of the sealing plug and container.

FIG. 45 illustrates a cross sectional view of one embodiment of the present invention of a collection container with a coating, film or label on the outer surface of the interface of the sealing plug and container.

FIG. 46 illustrates a partial cut away view of the present invention wherein the sealing plug of a collection container includes a sensor which is accessible to both the inside and the outside of the container.

FIG. 47 illustrates another embodiment of the present invention with a sealing plug and a diverting component.

FIG. 48 illustrates another embodiment of the present invention with a sealing plug and a diverting component with an internal well.

FIG. 49 illustrates a side view of a diverting component that may be joined to a sealing plug of the present invention.

FIG. 50 illustrates a bottom view of a diverting component which shown in FIG. 49.

FIG. 51 illustrates a cross sectional side view of a sealing plug in one embodiment of the present invention.

FIG. 52 illustrates a full bottom view of the sealing plug shown in FIG. 51.

FIG. 53 illustrates another embodiment of the present invention of a self-shielding sealing plug residing in an openable end of a container.

FIG. 54 illustrates a cross sectional view of the sealing plug of FIG. 53 with a self-shielding sealing plug removed from an openable end of a container with a slidable shield activated on a sealing plug.

FIG. 55 illustrates another embodiment of a sealing plug and shield with a flow indicating or viewing section.

FIG. 56 illustrates a cross sectional view of a collection container of the present invention with rounded ends.

FIG. 57 illustrates a cross sectional view of the present invention wherein a coupler or extension allows a large diameter collection container to be used with a needle holder having a diameter smaller than the larger diameter container.

FIG. 58 illustrates a cross sectional view of the present invention wherein a flow regulating coupler or extension allows a large diameter container to be used with a needle holder having a diameter smaller than the larger diameter container.

FIG. 59 illustrates cross sectional view of the present invention wherein a single collection container can be used with needle holders having different size diameters.

FIG. 60 illustrates a side view of a sealing plug of the present invention with a venting means.

FIG. 61 illustrates a full bottom view of the sealing plug of FIG. 60.

FIG. 62 illustrates a cross sectional view of a sealing plug shown in FIG. 60 closing a open end of a collection container.

FIG. 63 illustrates a cross sectional view of a sealing plug shown in FIG. 62 exposing the internal chamber of a collection container to the outside of a container prior to full removal of a sealing plug from a collection container.

FIG. 64 illustrates a cross sectional view of a sealing plug of the present invention with a sealed chamber having external access.

FIG. 65 illustrates a cross sectional view of the sealing plug of FIG. 64 with a chamber being open.

FIG. 66 illustrates a full side view a sealing plug of the present invention with a movable shield having an aperture.

FIG. 67 illustrates a full side view of the sealing plug of FIG. 66 with the aperture of a movable shield positioned adjacent to a chamber of the sealing plug.

FIG. 68 illustrates a cross sectional view of a sealing plug and a movable shield shown in FIG. 66.

FIG. 69 illustrates a cross sectional view of a sealing plug and movable shield of FIG. 67 with the aperture of a movable shield positioned adjacent to a chamber of a sealing plug.

FIG. 70 illustrates a cross sectional side view of a container with a sealing plug including a means to diffuse fluid entering the container.

FIG. 71 illustrates the blood collection apparatus of FIG. 70 with a specimen being diffused as it is introduced into the container.

FIG. 71A illustrates the blood collection apparatus of FIG. 71 showing a cross sectional side view of a needle lumen as a specimen is being introduced and diffused from the needle into the container .

FIG. 71B illustrates a blood collection apparatus having a regulating means within a needle lumen to regulate or diffuse the fluid flowing through the needle and into a container.

FIG. 72 illustrates a partial, cross sectional side view of a container with a sealing plug including an aperture with a liquid activated section.

FIG. 73 illustrates a cross sectional side view of the container of FIG. 72 containing a liquid with the aperture closed at the liquid activated section.

FIG. 74 illustrates a cross sectional view of the present invention with a diverting means combined with a prior art sealing plug. The diverting component may also include a means to improve the seal at the sealing plug/container surface interface.

FIG. 75 illustrates a cross sectional view of the present invention with a means to improve the seal at the sealing plug/container surface interface.

FIG. 76 illustrates a cross sectional view of the present invention having a sealing plug with a pressure sensitive valve which is activated when a pressure difference exists on either side of the valve.

FIG. 77 is a full side view of the valve component of FIG. 76.

FIG. 78 illustrates a cross sectional side view of a sealing plug with a diverter having an open channel or slot for accessing the specimen in a container with a long needle without having to remove the sealing plug.

FIG. 79 illustrates a cross sectional top view of the diverter of FIG. 78 having an open channel or slot and a diverter chamber separated by a wall section.

FIG. 80 illustrates a blood collection needle with a needle guard which can only be activated manually.

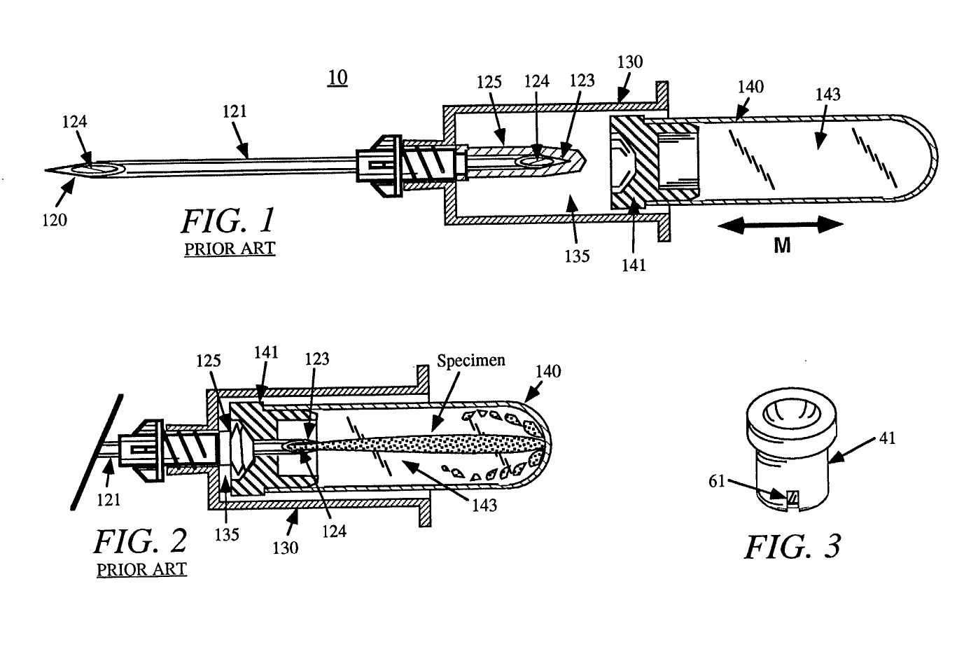

### DETAILED DESCRIPTION OF THE INVENTION

An apparatus and method for collecting a blood specimen or other bodily fluid or gaseous substance is described. In the following description, numerous specific details are set forth, such as material types, dimensions, processes, etc., in order to provide a thorough understanding of the present invention. However, it will be obvious to one of ordinary skill in the art that the invention may be practiced without these specific details. In other instances, well-known structures and processing steps have not been shown in particular detail in order to avoid unnecessarily obscuring the present invention. Additionally, it should be noted that this discussion will focus primarily on the collection of blood from a human patient. It should be understood, however, that such focus is for illustrative purposes only. The present invention is not limited to blood collection nor is it limited to the collection of a specimen sample from a human patient.

The collection container used to collect a specimen, referred to as 40 throughout this application, is made of glass, plastic resin, or a composite material and is normally evacuated and closed by a puncturable sealing plug. The internal chamber, referred to as 43 throughout this application, is the void or chamber within the container 40.

Referring to FIG. 3, a full side view of a sealing plug 41 having a chamber 61 openly connected to the side of the sealing plug. Sealing plug 41 is insertable into the open end of a container.

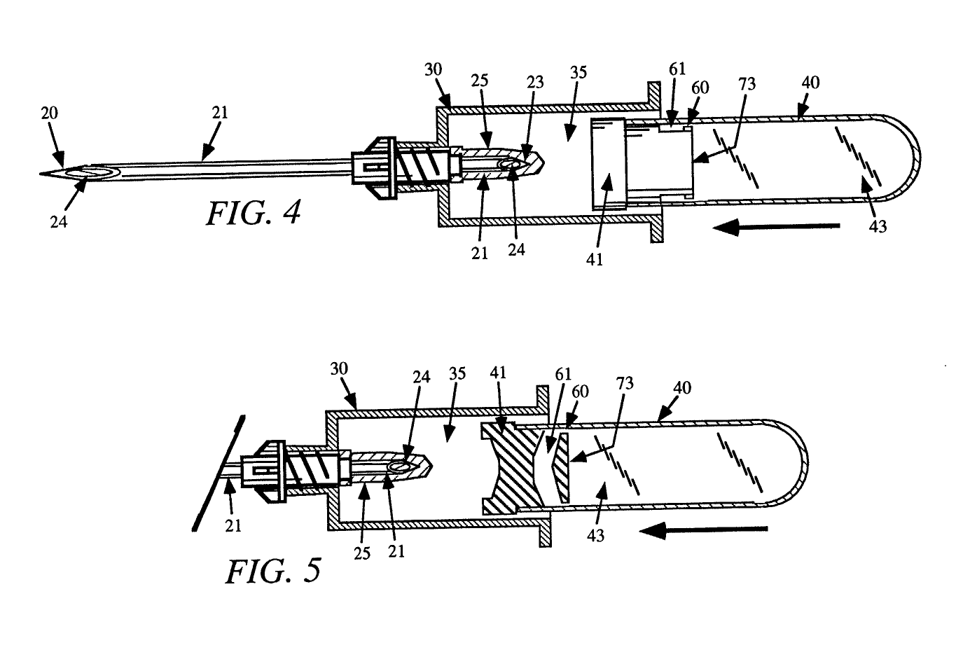

FIG. 4 is a full view of a blood collection needle 21 and sealing plug 41 are shown, and a cross sectional view of a needle holder 30, proximal needle cover 25 and container 40 are shown. Collection tube 40 has an open end and closed end, with inner cavity 43 created by placing sealing plug 41 in the open end for maintaining a sub-atmospheric pressure within the tube.

Piercable sealing plug 41 comprises a diverting means 73 and at least one chamber 61 for diverting a specimen flow as it enters the sealing plug. Chamber 61 is connected to internal chamber 43 of container 40 by at least one passageway 60. Container 40 is positioned in a cavity 35 of needle holder 30. Needle 21 has a sharpened, distal end 20 and proximal end 23. Distal end 20 is insertable into a blood vessel to obtain a specimen sample of blood for examination. Proximal end 23 resides within needle holder 30 and pierces sealing plug 41 during the collection procedure allowing specimen to flow from a blood vessel and into container 40. Needle 21 includes a lumen 24 communicating openly with each end creating a passageway from proximal end 23 to distal end 20. A needle cover 25 is typically provided to seal the proximal end of the needle 21.

Sealing plug 41 automatically diverts the specimen from its original fluid path when the sealing plug 41 is initially punctured by the needle 21, eliminating the probability of the specimen gaining momentum as it enters container 40. the size of passageway 60 may be lesser, equal to, or greater than the area of the needle lumen. A passageway 60 equal to or greater than the area of the needle lumen allows the specimen to flow freely. A passageway 60 smaller than the area of the needle lumen reduces and regulates the specimen with no change in the collection process.

FIG. 5 shows a cross sectional view of container 40, sealing plug 41 and needle holder 30 of FIG. 4 with container 40 positioned within cavity 35 of needle holder 30. Sealing plug 41 includes at least one chamber 61 for diverting a specimen flow. In one embodiment, chamber 61 includes a diverting means 73 in the front of a convex wall section which regulates specimen flow from a patient to a collection container 40. Convex wall section allows any specimen remaining in chamber 61 to flow into container 40 prior to, or during the centrifuge process.

At least one passageway or aperture 60 is provided between chamber 61 to provide fluid communication between chamber 61 and tube cavity 43. Diverter 73 can include a dissolvable material, which diverts the specimen during the collection process and dissolves after the specimen is collected. A dissolvable material may also be used which facilitates analysis of the collected specimen.

A smaller or reduced passageway 60 is capable of regulating the volume of specimen being collected to prevent vein collapse associated with standard blood collection procedures. Different passageway 60 sizes would allow a variety of flow collection rates to be achieved without changing any currently used techniques.

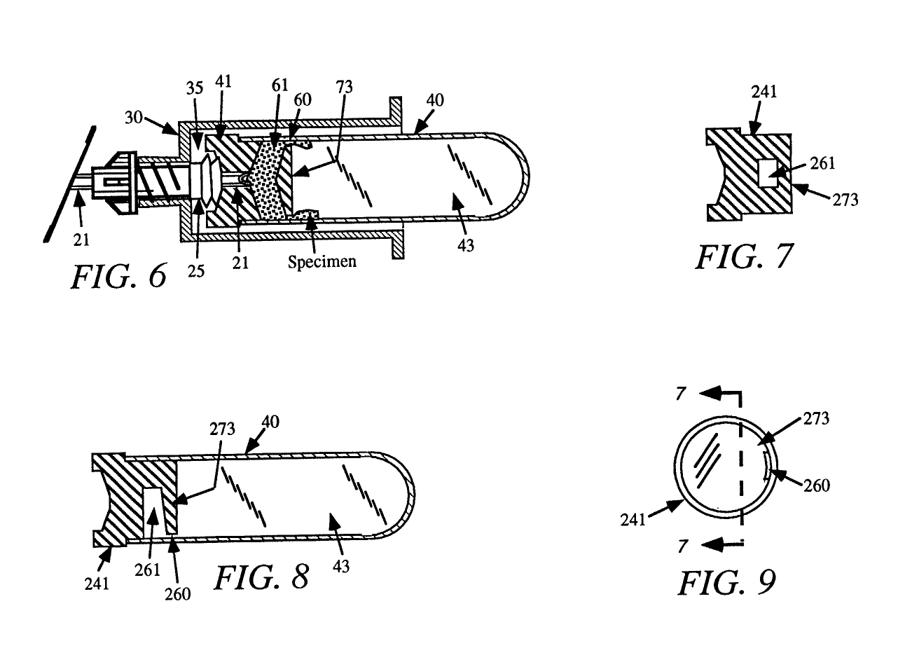

FIG. 6 is a cross sectional view of FIGS. 4 and 5 showing a specimen being introduced first into chamber 61 of sealing plug 41 and then into container 40. Specimen flow is diverted from needle 21 prior to entering internal cavity 43 of collection container 40 by diverter 73, chamber 61 and passageway 60. Sealing plug 41 maintains specimen within container 41 after collection, keeping the specimen free of contamination.

FIG. 7 is a cross sectional view of a sealing plug shown in axis 7-7 in FIG. 9 used to close a collection container, comprising chamber 261 and diverter 273 for regulating, governing, diverting, reducing, increasing, re-directing or interrupting the specimen flow through, into or within sealing plug 241 during blood collection procedures, chamber 261 is positioned adjacent to recess 260.

FIG. 8 is a cross sectional view of a container 40 of the present invention having one open end, a closed end and an internal chamber 43. The open end being sealingly closed by removable sealing plug 241 with a diverter 273, at least one chamber 261 and at least one recess or void 260 creating an inner channel, port or passageway when sealing plug 241 is positioned within open end of container or evacuated tube 40. The sealing plug 241 and chamber 261 could also have a smaller cross sectional thickness to reduce the overall mass of the plug. By reducing the size and mass of the sealing plug, manufacturing costs are lowered.

The inner wall of container or collection tube 40 or chamber 261 may also include a coating, additive, gel, inert polymer, or other substances which are used in the normal course of collecting and analyzing blood and blood products.

FIG. 9 is a full bottom view of the sealing plug of FIG. 7 comprising a sealing plug 241, used to close a container, having a diverter 273 for regulating, governing, diverting, reducing, increasing, re-directing or interrupting specimen flow through, into or within sealing plug 241 and through at least one recess 260 during blood collection procedures, recess 260 creates an inner channel or passageway when sealing plug 241 is positioned in or within the open end of container or evacuated tube 40. Sealing plug 241 may include a passageway from chamber directly through the bottom of diverter 273, eliminating the need to be positioned in or within the open end of container or evacuated tube to create a passageway.

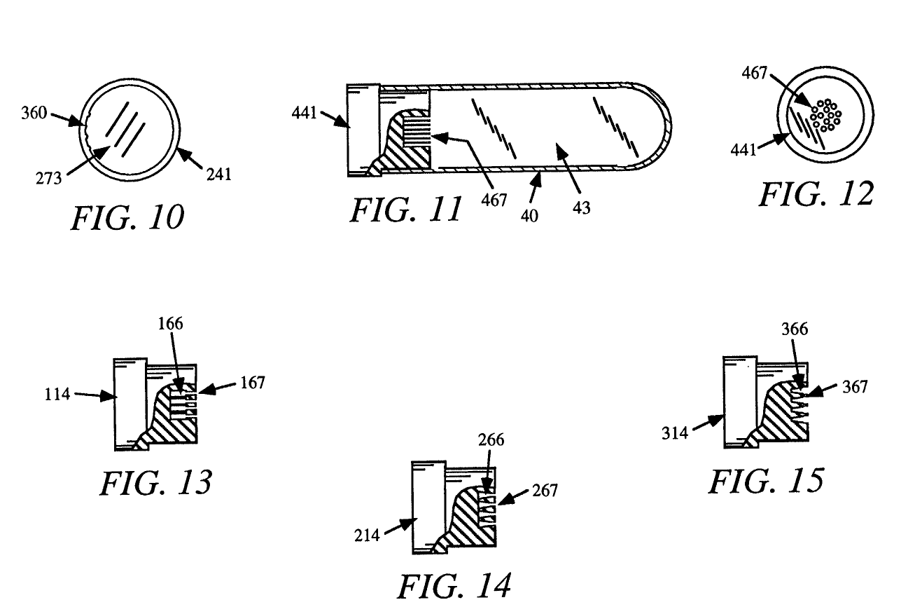

FIG. 10 is a full bottom view of another embodiment of a sealing plug 241, used to close a container, having a diverter 273, and a plurality of recesses, ports or channels 360 for regulating, diverting, re-directing, reducing, increasing or interrupting the specimen flow through, into or within sealing plug 241 during blood collection procedures. Recess 360 creates an inner channel or passageway when sealing plug 241 is inserted in or within the open end of container or evacuated tube.

FIG. 11 is a cross sectional side view of a collection container 40 with an internal chamber 43, with a cut away view of a sealing plug 441, used to close container 40, having a plurality of uniformly sized distal apertures or channels 467 for regulating, controlling or slowing the specimen flow through, into or within sealing plug 441 before the specimen enters container 40. Although apertures 467 are shown having a uniform shape here, they may be tapered, irregular, contoured or the like.

FIG. 12 is a full bottom view of the sealing plug of FIG. 11 comprising sealing plug 441, which may include an elastomeric material sufficient to frictionally engage and seal an internal chamber of a container, and at least one section 467 to regulate, control or slow specimen flow through, into or within sealing plug 441. Regulating means 467 may comprise a honeycombed, specific or random pattern.

FIG. 13 is a partial cut away side view of a sealing plug of the present invention showing a flow regulating means comprising sealing plug 114, having a plurality of internal chambers 166 for accepting the end of a tube or needle which pierces sealing plug 114 and enters chambers 166, a reduced distal aperture 167 for regulating or slowing the specimen flow through, into or within sealing plug 114 before the specimen enters a connected collection container. Although internal chambers 166 are shown at one depth here, they may be positioned or staggered at different levels or depths to facilitate core extraction during manufacturing.

FIG. 14 is a partial cut away side view of a sealing plug of the present invention used to close a collection container, showing a flow controlling means comprising sealing plug 214, having a plurality of chambers 266 for accepting the end of a tube or needle which pierces sealing plug 214 and enters chambers 266. Reduced distal apertures 267 for regulating or slowing the specimen flow through, into or within sealing plug 214 before the specimen enters a connected collection container. Although internal chambers 266 are shown at one depth here, they may be positioned or staggered at different levels or depths to facilitate core extraction during manufacturing.

FIG. 15 is a partial cut away view of sealing plug of the present invention used to close a container showing a flow regulating means comprising a sealing plug 314, having a plurality of chambers 366 for accepting the end of a tube or needle which pierces sealing plug 314 and enters chambers 366, increasing or expanding distal apertures 367 for regulating, slowing or increasing specimen flow through, into or within sealing plug 314 before the specimen enters a collection container. Although internal chambers 366 are shown at one depth here, they may be positioned or staggered at different levels or depths to facilitate core extraction during manufacturing. The specimen flow increases as the needle is advanced further into or through increasing chambers 366.

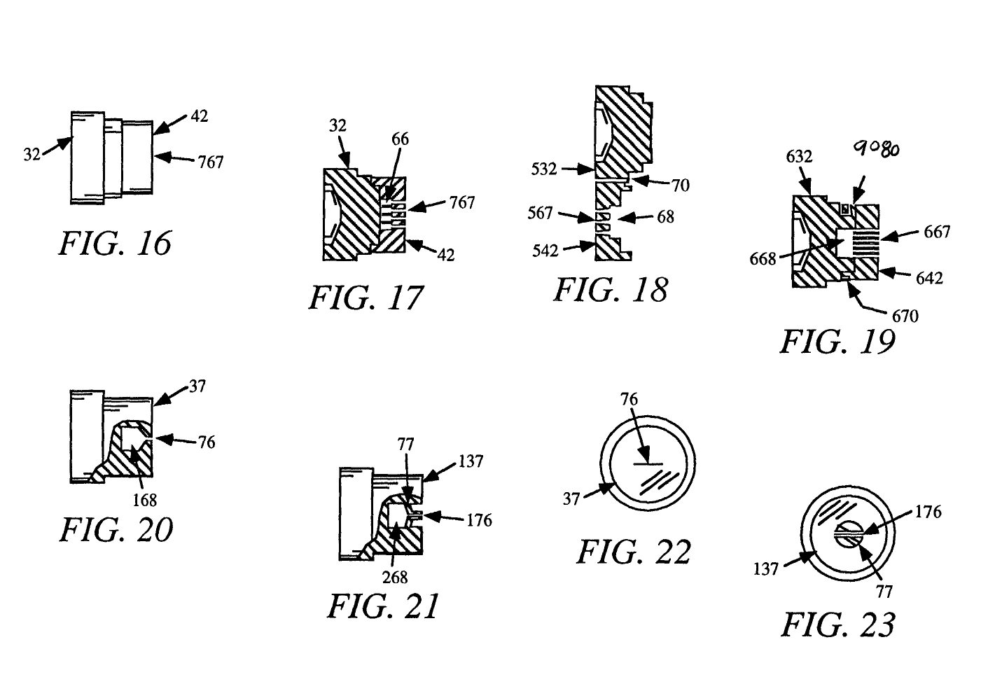

FIG. 16 is a full side view of a two-piece sealing plug of the present invention used to close a container having a piercable section 32 and a flow regulating or diverting section 42. Flow diverting section 42 having at least one aperture 767 for regulating, diverting or slowing the specimen flow through, into or within sealing plug 32 as specimen exits needle. Section 42 being slightly smaller in diameter or size than section 32 for easy removal of both sections 32 and 42 from a collection container.

FIG. 17 is a cross sectional side view of the two-piece sealing plug shown in FIG. 16 having a piercable section 32 and a flow regulating section 42. Flow regulating section 42 having a plurality of internal chambers 66 for accepting the end of a tube or needle which pierces sealing plug 32 and enters chambers 66. Chambers 66 are connected to a plurality of distal apertures 767 for regulating or controlling specimen flow through, into or within sealing plug 42 Although internal chambers 66 are shown at one depth here, they may be positioned or staggered at different levels or depths to facilitate core extraction during manufacturing.

FIG. 18 is a cross sectional side view of a two section sealing plug of the present invention having a joinable sealing plug 532 used to close a container. Sealing plug 532 is shown in an open faced configuration having two connected sections with piercable section 532, connected to a flow regulating section 542 having a recess 68 for creating an internal chamber when sealing plug sections 532 and 542 are joined together, and connecting means 70. Flow controlling section 542 having at least one recessed chamber 68 for accepting the end of a tube or needle which pierces sealing plug 532 and enters the chamber 68. Chamber 68 is connected to a plurality of distal apertures 567 for regulating or diverting the specimen flow through, into or within sealing plug 542 as specimen exits needle.

Although internal chamber 68 is shown at one depth here, it may be manufactured having different levels or depths. This open faced embodiment of the flow controlling sealing plug 542 allows a wide variety of flow controlling features to be easily incorporated into the invention during manufacture, including, but not limited to, reducing, increasing, tapered or contoured shapes of distal aperture 567.

FIG. 19 is a cross sectional side view of a two part sealing plug 632 shown in FIG. 18, now shown in a joined configuration having two connected sections 632 and 642, with one piercable section 632 having recess 668 for creating a chamber when sealing plug sections 632 and 642 are joined together, a connected piercable, flow controlling section 642, and connecting means 670. Piercable sealing plug 632 having at least one internal recessed chamber 668 for accepting the end of a tube or needle which pierces sealing plug 632 and enters chamber 668. Flow controlling section 642 having a plurality of distal apertures 667 for controlling or diverting the specimen flow through, into or within sealing plug 642 as specimen exits needle.

Although internal recessed chamber 668 is shown at one depth here, it may be manufactured having different levels or depths. Sealing plug sections 632 and 642 join together, shown here having a male pin or post 9079 into a female aperture or section. Joining section may also comprise an undercut, or other means for fixedly attaching or joining sealing plug sections 632 and 642 together.

FIG. 20 is a partial cut away view of a sealing plug of the present invention comprising one-piece, puncturable sealing plug 37 used to close a collection container showing a flow controlling means with intermediate chamber 168 and flow controlling reed valve 76.

FIG. 21 is a partial cut away view of a sealing plug of the present invention comprising a one-piece, puncturable sealing plug 137 used to close a container showing a recessed flow controlling means including a chamber 268, piercable wall section 77 and flow controlling reed valve 176. As the needle is advanced into chamber 268, reed valve 176 allows the specimen to flow into a container. The needle can be advanced further to pierce wall section 77, allowing a direct specimen flow into a container during the collection procedure.

FIG. 22 is a full bottom view of the sealing plug shown in FIG. 20 comprising sealing cap 37 with a reed valve 76.

FIG. 23 is a full bottom view of the sealing plug shown in FIG. 21 comprising sealing cap 137 with a recessed chamber wall 77 and reed valve 176.

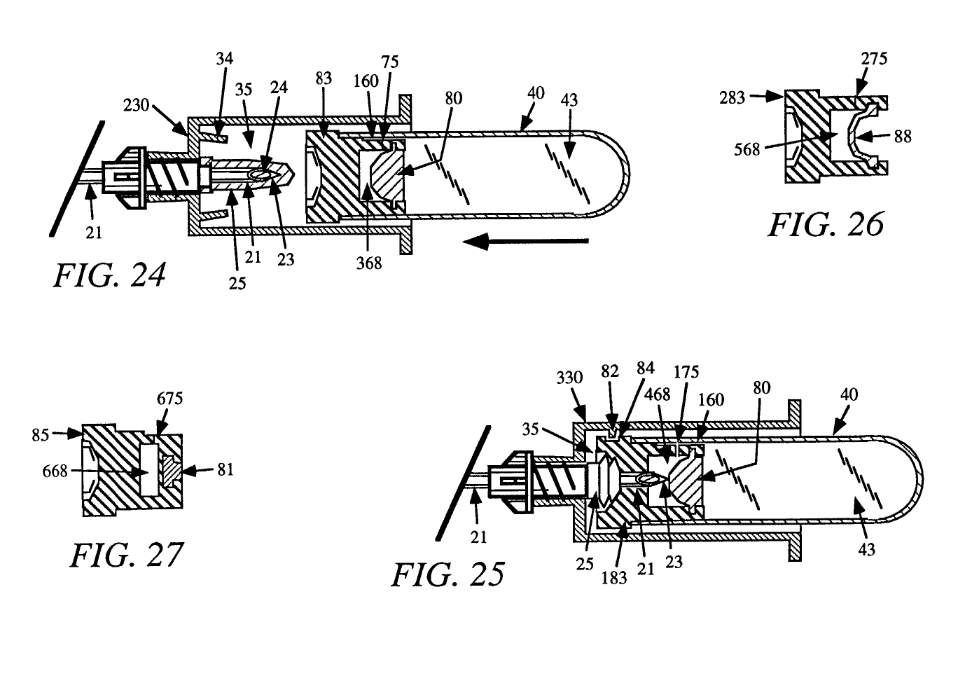

FIG. 24 is a cross sectional view of a blood collecting apparatus of the present invention prior to insertion showing a full view of needle 21, a needle holder 230 and container 40 having an internal cavity 43 with an adjustable flow, puncturable sealing plug 83, internal chamber 368, openable valve or port 75 and movable, substantially impenetrable secondary plug or diverter 80. Port 75 is shown here closed and adjacent to port or passageway 160. Needle holder 230 having a plurality of projections 34 for compressively resisting axial movement of container 40. A compressive force must be placed on container 40 in holder 230 to collect specimen.

FIG. 25 is a cross sectional view of the blood collecting apparatus of the present invention during the collection process comprising needle 21, needle holder 330 and container 40 having an internal cavity 43 with an adjustable flow, puncturable sealing plug 183, internal chamber 468, openable valve or port 175 and movable, substantially impenetrable secondary plug 80. As needle 21 enters chamber 468, needle tip 23 engages diverter 80 which opens port 175 allowing specimen to flow into inner cavity 43 of collection tube 40. Port 175 opening can be reduced by partially disengaging needle tip 23 from movable plug 80. Port 175 opening can be closed completely by fully disengaging needle tip 23 from secondary plug 80.

Penetration-related adjustable flow is rotationally controlled by frictional or threaded means 84 of sealing plug 183 and frictional or threaded means 82 of needle holder 330.

FIG. 26 is a cross sectional side view of an adjustable flow sealing plug of the present invention used to close a container, used in the same manner as the sealing plugs shown in FIGS. 25 and 26, comprising a puncturable sealing plug 283 having an intermediate chamber 568, openable port 275, and movable plug 88 with a uniformly consistent wall section.

FIG. 27 is a cross sectional side view of an adjustable flow, puncturable sealing plug of the present invention comprising a sealing plug 85 having an intermediate chamber 668, openable port 675, and integrally molded, substantially impenetrable section or stop 81, which is movable when a needle engages stop 81.

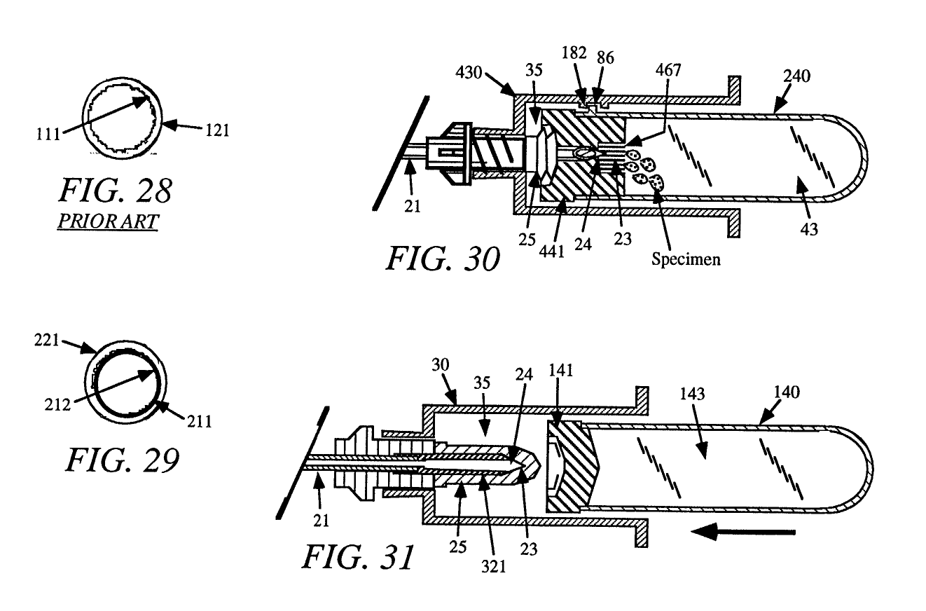

FIG. 28 is a cross sectional front view of a prior art hollow bore needle 121 having an outer smooth wall and rough inner wall 111.

FIG. 29 is a cross sectional front view of hollow bore needle of the present invention having an outer smooth wall, with an inner wall 211 being coated with friction reducing lubricant 212 to reduce the rough surface of the inner wall 211. Inner wall 211 may also be manufactured in a smooth fashion by mechanical or chemical means. Lubricant or filler 212 is deposited into recesses of inner wall 211, creating a smoother, inner wall surface. Inner wall coating 212 can also comprise a material which inhibits blood clotting.

FIG. 30 is a cross sectional view of a blood collecting apparatus of the present invention during the collection process comprising a needle 21, needle holder 430 and container 240 having an internal chamber 43 with an inserted sealing plug 441 having a plurality of uniformly sized distal apertures 467 for controlling or diverting the specimen flow through, into or within sealing plug 441 as the specimen exits the needle 21.

Penetration-related adjustable flow is controlled by rotational movement of container 240 relative to needle holder 430 by projection or thread 86 of container 240 and corresponding projection or thread 182 of needle holder 430. Penetration-related adjustable flow may be controlled by frictional engagement of container 240 and needle holder 430.

FIG. 31 is a cross sectional view of a blood collecting apparatus of the present invention prior to use comprising a needle 21 connected to enlarged bore needle 321, needle holder 30 and collection container 140 having inner cavity 143 and sealing plug 141.

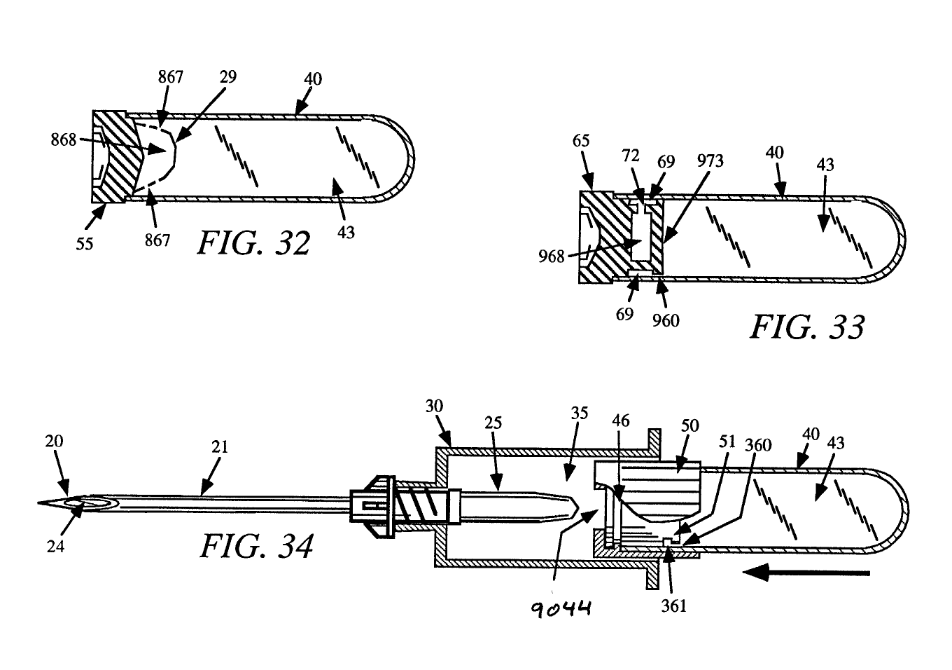

FIG. 32 is a cross sectional view of a container of the present invention showing a container 40 having inner cavity 43, a puncturable sealing plug 55 with separate membrane 29 with at least one aperture or port 867 whereby a membrane 29 creates intermediate chamber 868 adjacent to sealing plug 55. Membrane 29 can be permeable, or impermeable with at least on pre-pierced section 867.

FIG. 33 is a cross sectional view of a collection container of the present invention showing container 40 having inner cavity 43, with open end of container 40 being sealed by a multi-chambered sealing plug 65 with inner chamber 968 and secondary chamber 69 created by diverter 973. Chamber 69 is connected to first chamber 968 by port, aperture or passageway 72. Specimen flows first into chamber 968 then into chamber 69 through port or passageway 960 to inner cavity 43. Secondary chamber 69 is originally a recess or channel of sealing plug 65 and creates chamber 69 when sealing plug 65 is positioned in or within the open end of container or vacuum tube 40.

Sealing plug 65 has an external configuration similar to a standard syringe piston.

FIG. 34 illustrates a blood collection apparatus of the present invention prior to use with a full view needle 21 and sealing plug 51, with a cross sectional view of needle holder 30, tube 40 and a partial cut away view of shield 50, comprising a puncturable sealing plug 51 with inner chamber 361 and passageway 360 connected to internal chamber 43 of container 40. Sealing plug 51 comprises at least one intermediate chamber 361 for regulating or diverting specimen flow from a needle 21 to a container 40. Shield 50 is connected to sealing plug 51 at interface 46 for removal from container 40, reducing exposure of blood and bodily fluids to healthcare workers during collection and testing procedures. Shield 50 having a radially extending face covering top of shield 50 with aperture or opening 44 for accessing sealing plug 51 with needle 21. Sealing plug 51 is contained within shield 50 which extends annularly around sealing plug 51.

Shield 50 facilitates easy removal of sealing plug 51 from container 40. Container 40 is positioned in cavity 35 of needle holder 30. Distal end 20 of hollow bore needle 21 is insertable into a blood vessel to obtain a specimen sample of blood for examination.

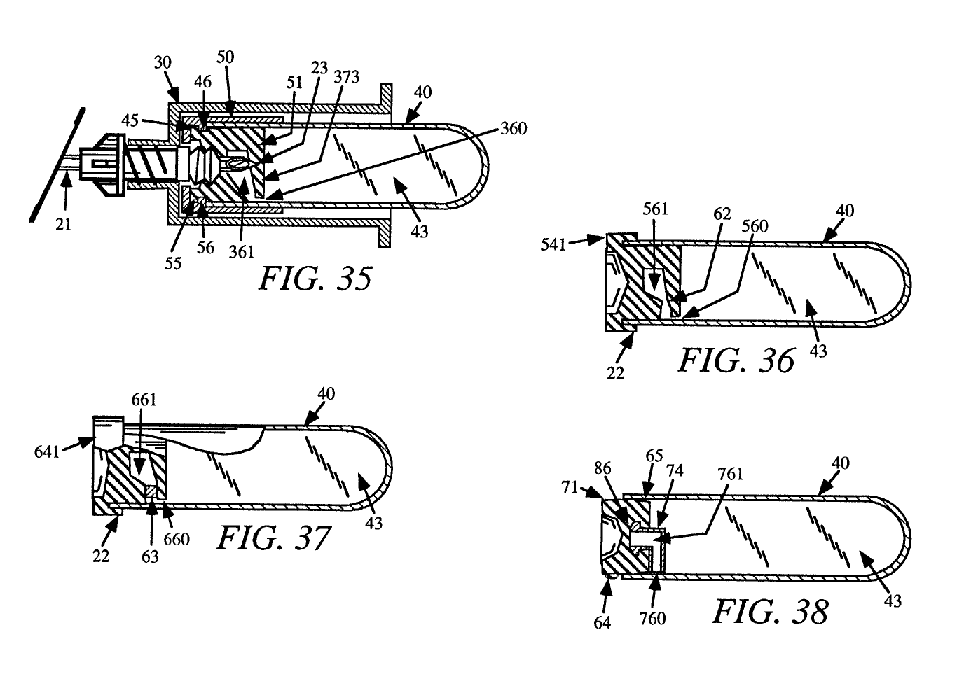

FIG. 35 shows a cross sectional side view of collection container of FIG. 34 during use with container 40 having sealing plug 51 with at least one intermediate chamber 361 for diverting or maintaining specimen flow in a fluid suspension as specimen is collected from a patient to container 40. At least one port or aperture 360 provides a passageway between chamber 361 of sealing plug 51 and internal cavity 43 of container 40. Sealing plug 51 having a shield 50 to facilitate easy removal of sealing plug 51 from container 40. Sealing plug 51 having at least one recess 46 which may be annular or intermittent for fittingly engaging sealing plug 51 and protrusion or lip 45. Shield 50 having a recess 55 for fittingly engaging protrusion 45 of sealing plug 51. Shield 50 having at least one projection 56 which may be annular or intermittent for attaching sealing plug 51.

Chamber 361 is manufactured in a pre-determined position whereby relationship of needle tip 23 penetrates only into chamber 361, and not directly into internal chamber 43 of container 40. Needle tip 23 could also be manufactured longer to penetrate intermediate chamber 361 first to allow specimen flow from needle 21, and with further axial advancement needle tip 23 could reside within diverting means 373, stopping specimen flow during the collection process, and finally needle tip 23 could also be manufactured to penetrate intermediate chamber 361 first to allow specimen flow from needle 21 and with further axial advancement needle tip 23 could reside within diverting means 373, stopping specimen flow during the collection process, and with even further axial advancement, needle tip 23 could penetrate through diverting means 373 and directly into internal chamber 43 of container 40, totally bypassing intermediate chamber 361 and diverting means 373 and allowing direct specimen flow from needle 21 into container 40.

FIG. 36 is a cross sectional side view of a collection container of the present invention showing container 40 having sealing plug 541, intermediate chamber 561 with reducing section 62 to control, regulate or divert specimen flow into container 40 with passageway 560 connecting intermediate chamber 561 to internal chamber 43. Sealing plug 541 having an extending outer section 22 for maintaining a sub-atmospheric pressure within container 40.

FIG. 37 is a partial cut away view of a collection container of the present invention having one open end and an internal chamber 43, with a sealing plug 641 used to close container 40. Sealing plug 641 having chamber 661 for regulating, governing, diverting, re-directing, reducing, increasing or interrupting the specimen flow through, into or within sealing plug 641 during blood collection procedures. Passageway 660 connects intermediate chamber 661 to internal chamber 43. Chamber 661 having filtering means 63 to filter or control specimen flow during blood collection procedures. Filtering means 63 may also comprise a dissolvable material. Sealing plug 641 having an extending outer section 22 for maintaining a sub-atmospheric pressure within container 40.

FIG. 38 is a cross sectional view of a collection container of the present invention comprising a container 40, having one open end and internal chamber 43, open end being sealingly closed by removable, puncturable sealing plug 71 with at least one diverting component 74 creating chamber 761 and at least one recess 760 for creating an inner channel, port or passageway to internal chamber 43 when sealing plug 71 is positioned within open end of container or evacuated tube 40. Diverter 74 having an angled lead-in section 86 for easy assembly of component 74 to sealing plug 71. Sealing plug 71 having a chamfered or tapering bottom perimeter 65 for easy insertion into open end of container 40.

Internal cavity 43, inner wall of the tube 40 or diverter 74 may also include a coating, additive, gel, inert polymer, or other substances which are used in the normal course of analyzing blood and blood products.

Smaller sized sealing plug 71 requires less puncturable material to close a collection container by adding another low-cost, diverting component 74 to create chamber 761 and passageway 760. Indicator 64 is shown on sealing plug 71 to determine the location of passageway 760 leading from sealing plug 71 into internal chamber 43 of container 40. Indicator 64 is shown directly adjacent to passageway 760 of sealing plug 71. Indicator 64 can also be placed at any position, or directly opposite, or 180° away from, the location of passageway 760.

Diverting component 74 may include a hinged section whereby diverting section is maintained in a diverting position during the normal collection procedure and is opened by the centrifugal force generated during the centrifuge process. This allows any specimen remaining within an intermediate chamber to be combined with the specimen in the internal chamber 43 of container 40.

Diverting component 74 may also re-direct specimen flow first toward the sidewall of container 40 and then directly into internal cavity 43, forming a “Z” like configuration.

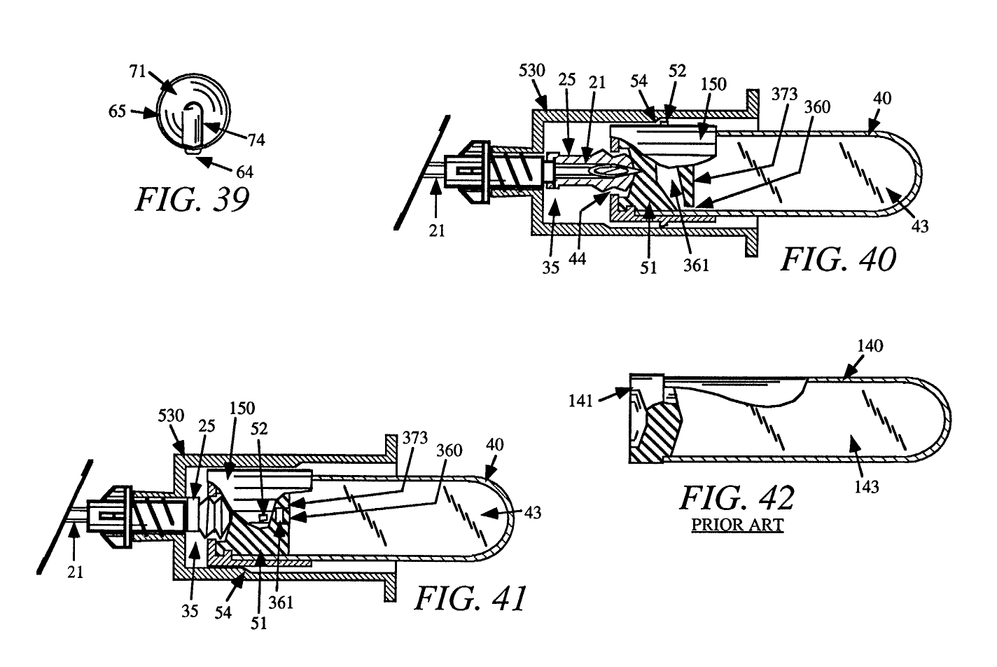

FIG. 39 is a full bottom view of the sealing plug shown in FIG. 38 comprising a sealing plug 71, with a chamfered section 65, an indicator 64, and a separate diverter 74. Diverter 74 may include a dissolvable material when activated by a wet solution like blood.

FIG. 40 is a full, cross sectional and partial cut away view of a blood collecting apparatus of the present invention showing sealing plug 51 being partially pierced by needle 21. The blood collection apparatus comprises a blood collection needle 21, a needle holder 530, and a container 40 with a sealing plug 51 and shield 150. Sealing plug 51 is used to close container 40, and has at least one intermediate chamber 361 and diverter 373 for regulating, diverting, re-directing, reducing, increasing or interrupting the specimen flow through, into or within sealing plug 51 during blood collection procedures. Recess 360 creates an inner channel or passageway when sealing plug 51 is positioned in or within the open end of container 40.

Shield 150 having an aperture 9044 for unrestricted access of needle 21 into sealing plug 51, and a projection 52 for frictionally or rotationally engaging needle holder 530 and internal open-faced projection 54. Projection 52 engages projection 54 during rotational movement of container 40 towards needle 21, maintaining a positive control during sealing plug 51 puncture by needle 21. Direct axial removal of container 40 is unrestricted due to the open-faced configuration of projection 54 of needle holder 530, allowing container 40 to be removed by a straight pulling movement like a standard collection container is now removed from a needle holder.

Penetration-related adjustable specimen flow is regulated by frictional or threaded engagement of projection 52 of container 40 or shield 150 and frictional or threaded engagement of projection 54 of needle holder 30.

FIG. 41 is a full and cut away view of a blood collecting apparatus illustrated in FIG. 40 showing the needle 21 fully piercing sealing plug 51, with container turned 90° in a rotational manner. The blood collection apparatus comprises blood collection needle 21, needle holder 530, and container 40 with sealing plug 51 and shield 150. Sealing plug 51 is used to close container 40, having at least one internal chamber 361 and diverter 373 for regulating, diverting, re-directing, reducing, increasing or interrupting the specimen flow through, into or within sealing plug 51 during blood collection procedures. Recess 360 creates an inner channel or passageway when sealing plug 51 is positioned in or within the open end of container 40.

Shield 50 having a projection 52 for frictionally or rotationally engaging needle holder 530 and internal projection 54. Projection 52 engages projection 54 during rotational or frictional movement of container 40 towards needle 21, maintaining a positive control during sealing plug 51 puncture by needle 21. Direct axial removal of container 40 is unrestricted due to the open faced configuration of projection 54 of needle holder 530 whereby container 40 can be removed from needle holder 530 by a straight pulling motion.

FIG. 42 is a partial cut away view of a prior art blood collection container 140 having a sealing plug 141 to maintain a sub-atmospheric pressure within internal chamber 143. Container 140 is normally comprised of either glass or shatter resistant plastic. The major limitation of using plastic as the container body 140 is the tendency of the collected blood to react unfavorably with the elements contained in the plastic resin. The inside of the plastic tube must be completely coated with an additional barrier or film to achieve the same compatibility as the glass substrate. This adds additional cost to the collection container.

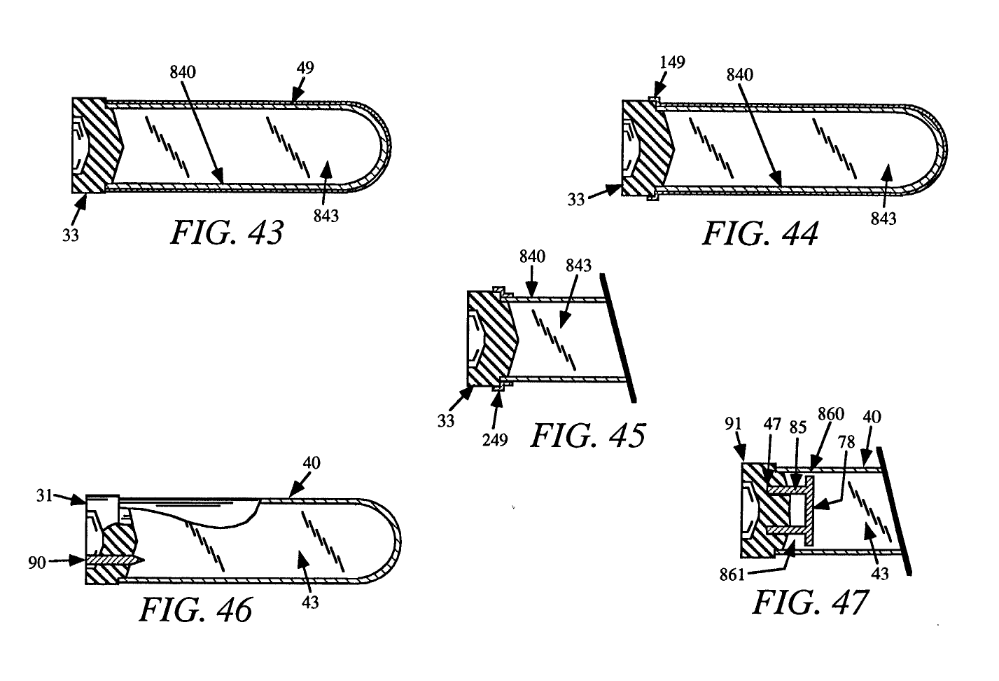

FIG. 43 is a cross sectional view of a collection container of the present invention comprising a container 840 having piercable sealing plug 33 to maintain a sub-atmospheric pressure within internal chamber 843 and coating or film 49 on the outer surface of container 840 to reduce shattering probability in the event container 840 is broken during manufacturing, storage or use. Coating or film 49 comprises a protective material, which bonds to the outside surface of container 840. Coating or film 49 keeps collection container 840 intact during manufacture, storage and use.

Coating or film 49 can include, but is not limited to, a polymeric or elastomeric material which can also be applied or sized by chemical, electrical or heat processes. Coating or film 49 maintains tube substrate in an integral fashion and houses specimen safely within container 840 when container is dropped or crushed, keeping the healthcare worker from being exposed to the blood or bodily fluid specimen and keeping the workplace safe. Film or coating 49 is applicable to any and all collection containers disclosed within this application.

FIG. 44 is a cross sectional view of a collection container of the present invention comprising a container 840 having piercable sealing plug 33 to maintain a sub-atmospheric pressure within internal chamber 843 and a coating or film 149 on the outer surface of container 840 extending over the juncture or interface where sealing plug 33 and container 840 join together. Coating or film 149 improves vacuum retention within internal chamber 843 and alerts healthcare worker if the seal has been tampered with if a tearing, stretching, or other deforming indication is present on coating or film 149 where vacuum could have been compromised.

FIG. 45 is a cross sectional view of the present invention comprising a container 840 having a sealing plug 33 to maintain a sub-atmospheric pressure within internal chamber 843 and a coating, film or label 249 over the juncture or interface where sealing plug 33 and container 840 join together. Coating, film or label 249 improves vacuum retention within internal chamber 843 and alerts healthcare worker if the seal has been tampered with if a tearing, stretching, or other deforming indication is present on coating, film or label 249 where vacuum could have been compromised.

FIG. 46 is a partial cut away view of a collection container of the present invention comprising a container 40 having a sealing plug 31 to maintain a sub-atmospheric pressure within internal chamber 43 and a sensor or probe 90 which is accessible from the outside of container 40. Specimen can be analyzed without removal of sealing plug 31 from container 40.

FIG. 47 is a partial cross sectional view of a collection container of the present invention comprising a container 40 having a sealing plug 91 to maintain a sub-atmospheric pressure within internal chamber 43 and a diverter 78, which can be comprised of a dissolvable or undissolvable material, creating an intermediate chamber 861 when attached to sealing plug 91, which re-directs specimen flow entering chamber 861 towards the outer perimeter o container 40. Diverter 78 is attached to sealing plug 91 by means of a plurality of projections 85 which engage sealing plug 91 at recess 47. Annular passageway 860 allows specimen flow to gravitate toward perimeter of collection container 40, or to the lower extremity of collection container 40 regardless of how container is positioned in a needle holder. A 360° specimen diversion, or any fraction thereof, is accomplished by diverter 78.

Diverter 78 is shown frictionally engaging recess 47 of sealing plug 91 whereby gripping force is sufficient to maintain attachment during the normal specimen collection procedure. All collected specimens are placed in a centrifuge and spun to separate the plasma from the red cells prior to testing. The gripping force of diverter 78 to sealing plug 91 is capable of releasing during the centrifuge process. This allows any specimen which remained within intermediate chamber 861 after the collection procedure to be combined with the specimen in the internal chamber 43.

Diverter 78 can also comprise a hinged section whereby diverting section is maintained in a diverting position during the normal collection procedure and is opened by the centrifugal force generated during the centrifuge process. This allows any specimen remaining within intermediate chamber 861 to be combined with the specimen in the internal chamber 43.

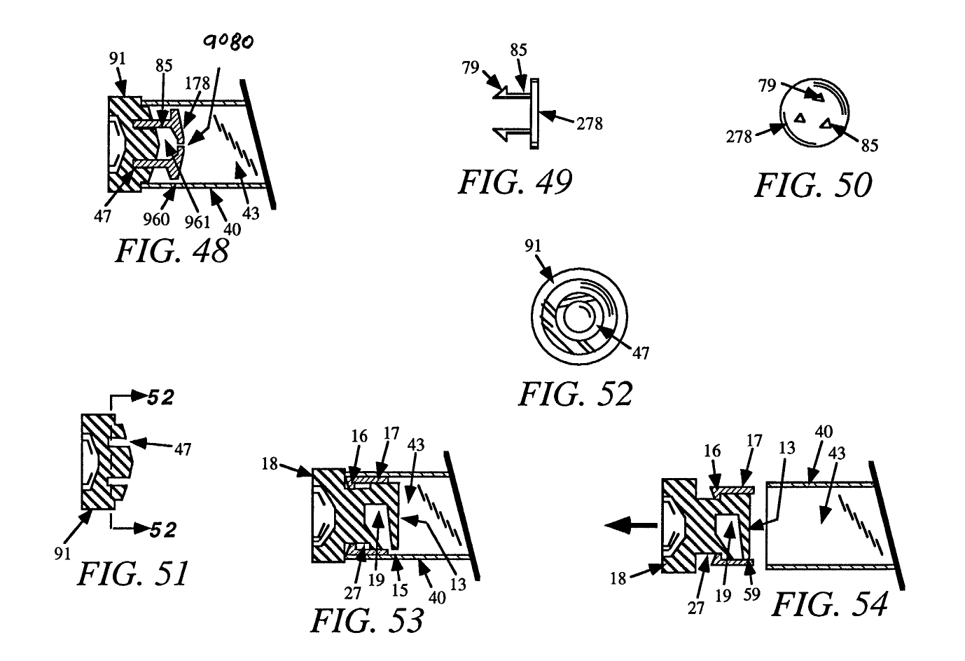

FIG. 48 is a partial cross sectional view of a collection container of the present invention comprising a container 40 having a sealing plug 91 to maintain a sub-atmospheric pressure within internal chamber 43 and a diverter 178 with a well which creates intermediate chamber 961 and re-directs a specimen flow entering chamber 961 towards the outer perimeter of container 40. Diverter 178 is attached to sealing plug 91 by means of a plurality of projections 85 which engage sealing plug 91 at recess 47. Diverter 178 having an aperture or channel 9079 for draining specimen within intermediate chamber 961 after collection.

Diverter 178 can be made of a dissolvable or undissolvable material. Aperture 9079 can include a dissolvable material which blocks aperture 9079 during the collection process and dissolves when exposed to liquid, opening aperture 9079 to allow any specimen contained in chamber 961 to empty into container 40. Annular passageway 960 allows specimen flow to gravitate toward perimeter of container 40 or to lower extremity of collection container 40.

FIG. 49 is a full side view of a diverter of the present invention which engages a sealing plug and creates intermediate chamber which re-directs specimen flow to the outer perimeter of a collection container. Diverter 278 having a plurality of projections 85 and barbs 79 which engage a sealing plug.

FIG. 50 is a full top view of the diverter shown in FIG. 49 having a plurality of projections 85 and barbs 79 extending from diverter 278.

FIG. 51 is a cross sectional side view of a sealing plug of the present invention comprising a piercable sealing plug 91 having at least one recess 47 for engaging a separate component. Recess may be annular, intermittent or the like to facilitate attachment of another component to sealing plug 91.

FIG. 52 is a full bottom view of the sealing plug shown in FIG. 51 in axis 52-52 having at least one recess 47 for engaging a separate component. Recess may be annular, intermittent or the like to facilitate attachment of another component to sealing plug 91.

FIG. 53 is a partial cross sectional view of a collection container of the present invention comprising self-shielding sealing plug 18 having an axially slidable shield 17 about one portion of shielding plug 18 inserted in an openable end of container 40. Puncturable sealing plug 18 having chamber 19 formed by diverter 13 for diverting specimen flow as it exits a needle, shown in other drawings. Passageway 15 openly connecting chamber 19 with internal chamber 43 of container 40.

Sealing plug 18 having an annular recess 27 for housing annular projection 16 for maintaining shield 17 in slidable engagement with sealing plug 18. Projection 16 is shown with chamfered top which allows easy, self-centering assembly of sealing plug 18 into shield 17 prior to insertion in openable end of container 40. Sealing plug and shield 17 comprise an air-tight seal to maintain a sub-atmospheric pressure within collection container 40.

FIG. 54 is a partial cross sectional view of the collection container shown in FIG. 53 with self-shielding sealing plug 18 and axially slidable shield 17 removed from openable end of container 40 with shield 17 automatically shielding portion of sealing plug 18 which was in contact with specimen in container 40. Shield 17 closing port 15 at intersection 59 of shield 17 and sealing plug 18 safely containing any specimen remaining within chamber 19 of sealing plug 18 from coming in contact with healthcare personnel. Specimen remaining in chamber 19 is unlikely after centrifuging container 40 with closed end of container 40 placed to the outer end of centrifuge. Puncturable sealing plug 18 having chamber 19 formed by diverter 13 for diverting specimen flow as it exits needle 21, shown in other drawings, and passageway or port 15 in communication with chamber 19 and internal chamber 43 of collection container 40. Projection 16 of shield 17 limits axial movement of sealing plug 18 as collection container 40 is opened. It is not necessary for shield 17 to close port 15 when shield 17 is moved to a protective position, as long as outer wall of diverter 13 is shielded by shield 17. Sealing plug and shield 17 comprise an air-tight seal to maintain a sub-atmospheric pressure within collection container 40.

When sealing plug 18 is removed from container 40, a greater gripping force between container 40 inner wall and sliding shield 17 outer wall allow sealing plug 18 to slide first in an axial manner in shield 17. Sealing plug 18 then is limited in axial movement within shield 17 by projection 16 whereby continued axial force removes both sealing plug 18 and shield 17 from openable end of container 40.

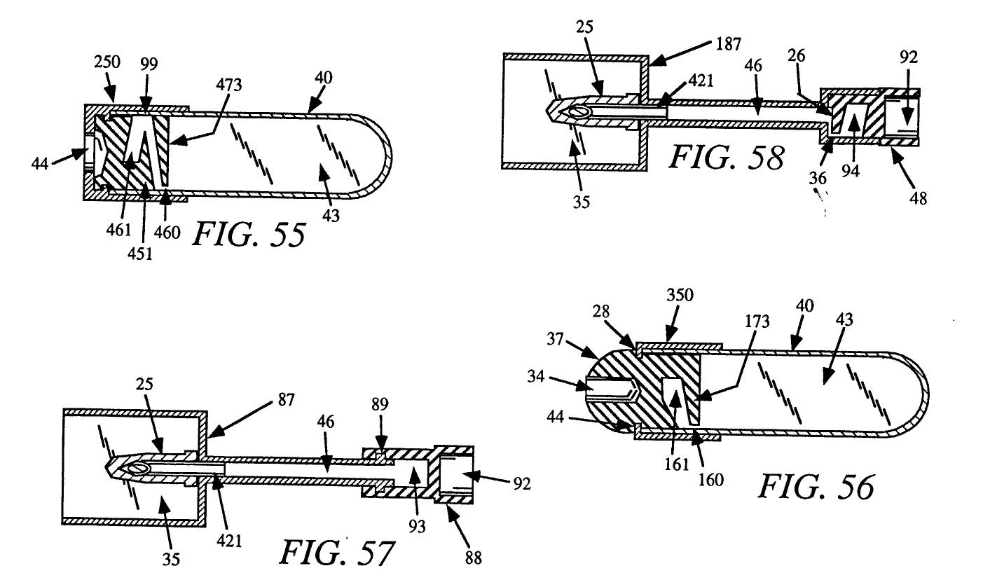

FIG. 55 is a cross sectional view of a collection container of the present invention comprising a translucent, flexible shield 250 and sealing plug 451 having a flow indicator or viewing area 99 to determine specimen flow into chamber 461. Specimen flow is diverted during the collection process by chamber 461 and diverter 473, and through passageway 460 into cavity 43 of container 40.

Since the container 40 and shield 250 are made of clear or translucent materials, specimen flow is easily observed. Shield 250 is not a necessary component whereby specimen flow is viewable through clear container 40 wall when sealing plug 451 is used individually. Flow Chamber 461 is easily manufactured by standard injection molding methods. It is preferable to have shield 250 as clear as possible for easiest viewing of specimen flow.

FIG. 56 is a cross sectional view of a collection container of the present invention comprising container 40 with an internal cavity 43, which may be evacuated, closed by sealing plug 37 with a semi-circular outer perimeter allowing either end of container 40 to be placed in a centrifuge. Tubular shield 350 having at least one projection 28 for engaging at least one recess 44 of sealing plug 37. Shield 350 has no top face and extends around the inserted sealing plug 37 with chamber 161 and diverter 173 to contain specimen within sealing plug 37 and shield 350 when both are removed from container 40. Recessed well 34 creates a smaller puncturable section of sealing plug 37 for easy insertion of needle through sealing plug 37. Sealing plug 37 having a diverter 173, chamber 161 and passageway 160 are connected with internal chamber 43 of container 40. Container 40 can include a closed end configuration having a square, geometric, oval or other non-circular shape.

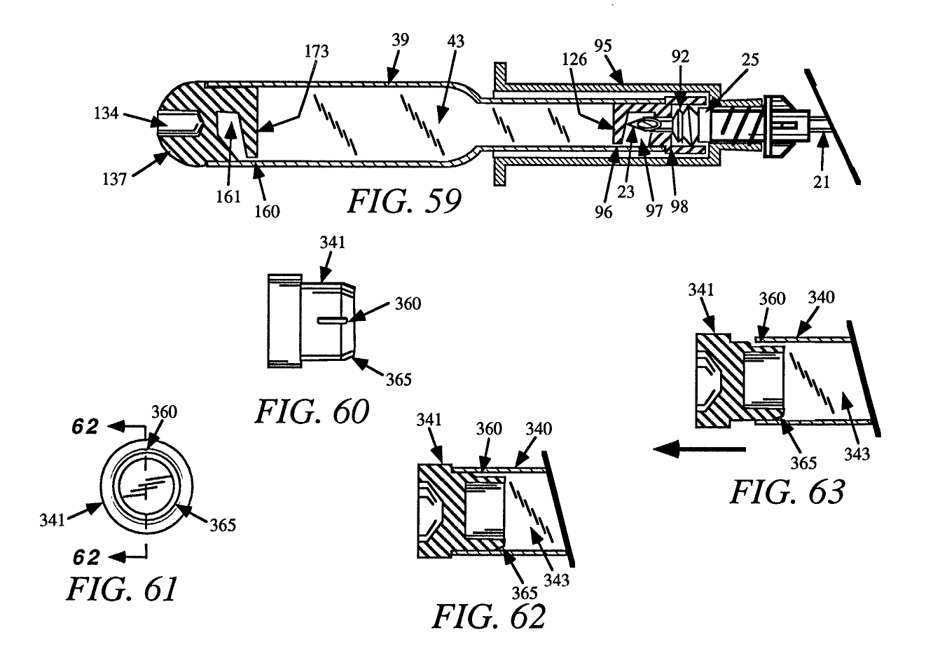

FIG. 57 is a cross sectional view of a blood collection adapter of the present invention comprising an extension or coupler 87 where a standard, or larger container 40, shown throughout this application, is usable with smaller, pediatric needle holder 95, shown in FIG. 59. Smaller diameter needle holder 95 allows a shallower angle to be used to access a blood vessel during blood collection procedures.

A hollow bore needle, attached to smaller diameter needle holder 95, is inserted into a blood vessel and sealing plug 88 end of coupler 87 is inserted into needle holder 95. Cover 25 contains specimen within chambers 46 and 93, and needle 421 until a larger diameter collection container is slid into coupler 87 and sealing plug of larger diameter container is pierced. A larger diameter collection container can be used to collect a specimen using a smaller diameter needle holder 95, shown in FIG. 59. The smaller diameter needle holder allows a shallower angle to be used to access a blood vessel because the center point of needle holder 95 is closer to body surface of the patient. Coupler 87 is attached to piercable cap 88 at interface 89. Smaller diameter sealing plug 48 having an external well 92 to prevent residual specimen from coming in contact with healthcare personnel during specimen collection and analysis.

FIG. 58 is a cross sectional view of a blood collection adapter of the present invention comprising an extension or coupler 187 with a flow regulating plug 48 where a standard, or larger collection container, shown throughout this application, is usable with smaller diameter, pediatric needle holder 95. Plug 48 having chamber 94, diverter 26 and passageway 36 created by inserting plug 48 into coupler 187.

A hollow bore needle, attached to smaller diameter needle holder 95, is inserted into a blood vessel and sealing plug 48 end of coupler 187 is inserted into needle holder 95. Cover 25 contains specimen within chambers 46 and 94 and needle 421 until a larger diameter collection container is slid into coupler 187 and sealing plug of a larger diameter container is pierced. A larger diameter container 40 can be used to collect a specimen. using a smaller diameter needle holder 95. Smaller diameter needle holder 95 allows a shallower angle to be used to access a blood vessel because the center point of needle is closer to body surface of the patient. Smaller diameter sealing plug 88 having an external well 92 to prevent residual specimen from coming in contact with healthcare personnel during specimen collection and analysis.

FIG. 59 is a cross sectional view of a blood collecting apparatus of the present invention comprising container 39 capable of being used with either the standard needle holder, shown as a needle holder, or smaller diameter, pediatric needle holder 95. Smaller diameter needle holder 95 allows a shallower angle to be used to access a blood vessel because the center point of needle is closer to body surface of the patient. Smaller diameter sealing plug 98 having chamber 97, diverter 126, and passageway 96 for diverting specimen exiting from needle 21 into container 39. Larger diameter, opposite end of container 39 can be safely placed in a centrifuge to separate red cells from the plasma. Smaller diameter sealing plug 98 having an external well 92 to prevent residual specimen from coming in contact with healthcare personnel during specimen collection and analysis.

Larger diameter sealing plug 137 having chamber 161, diverter 173, and passageway 160 for diverting specimen exiting from a needle into container 39 Larger diameter sealing plug 137 having an external well 134 to prevent residual specimen from coming in contact with healthcare personnel during specimen collection and analysis. Container 39 can include just one open end whereby the opposite end would be closed.

FIG. 60 is a full side view of sealing plug of the present invention having a channel 360 for equalizing the internal pressure within the container with the ambient atmospheric pressure prior to full removal of sealing plug 341 from a container. Chamfer 365 aids assembly of sealing plug 341 into the open end of a collection container.

FIG. 61 is a full bottom view of the sealing plug shown in FIG. 60 having a channel 360 for equalizing the internal pressure within the collection container with the ambient atmospheric pressure prior to full removal of sealing plug 341 from a container. Chamfer 365 aids assembly of sealing plug 341 into the open end of a container.

FIG. 62 is a cross sectional view of sealing plug shown in FIGS. 60 and 61 with sealing plug 341 with channel 360 and chamfered bottom 365 inserted into container 340. Channel 360 and chamfered bottom 365 closing container 340 creating an internal chamber 343.

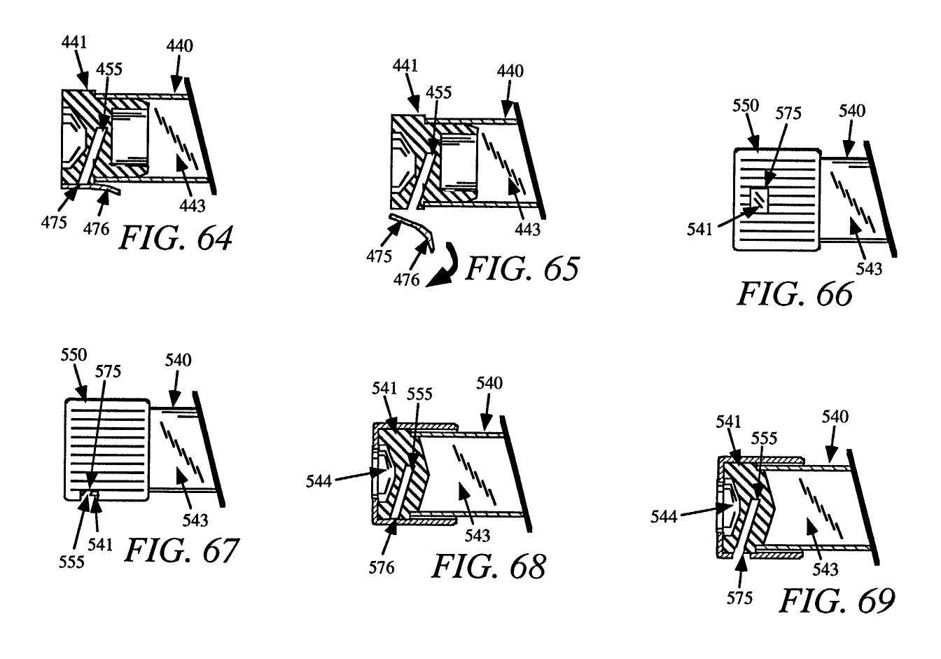

FIG. 63 is a cross sectional view of the sealing plug shown in FIG. 62 with a channel 360 and chamfered bottom 365, being moved from a sealing position and equalizing the internal pressure within the container 340 and chamber 343 with the ambient atmospheric pressure prior to full removal of sealing plug 341 from container 340. The equalization of internal pressure of chamber 343 reduces exposure probability to specimen contained within container 340.

FIG. 64 is a cross sectional side view of collection container of the present invention being closed by removable sealing plug 441 with an externally accessible chamber 455 sealed by strip 475 with pull tab 476. As a needle punctures sealing plug 441, hollow bore of needle containing specimen enters chamber 455 depositing a small amount of specimen within chamber 455. Specimen is collected in a normal fashion into chamber 443. When needle is removed from sealing plug 441, again a small amount of specimen is deposited within chamber 455.

Container 440 does not have to be opened, or does sealing plug 441 have to be punctured with a needle, or removed to obtain a small amount of specimen for visual analysis.

It is dangerous to withdraw the needle uncovered and deposit the collected specimen from the sharpened tip of the needle to a slide. With the probability of the needle being covered immediately upon withdrawal from a venipuncture site to prevent a needlestick accident, this invention makes it possible to obtain a small amount of collected specimen for a slide without exposing the healthcare worker to a sharp needle with blood in or on it.

Chamber 455 can be coated with an anti-clotting agent, dye or the like to facilitate visual examination of the specimen.

FIG. 65 is a cross sectional side view of collection container shown in FIG. 64 being closed by removable sealing plug 441 with an externally accessible chamber 455 being opened by removal of strip 475 with pull tab 476. Specimen deposited in chamber 455 can now be deposited onto a slide for visual examination.

FIG. 66 is a full side view of a collection container of the present invention with an externally accessible chamber 543 being closed by separable and movable sealing plug 541, with a movable external shield 550 having an aperture 575 closing container 540 creating internal cavity 543. External shield 550 is in a first position closing external access to chamber 555 of sealing plug 541.

FIG. 67 is a full side view of a collection container shown in FIG. 66 being closed by removable sealing plug 541 with movable external shield 550. Aperture 575 is now in a second position, relative to the first movable position, exposing chamber 555 allowing access to collected specimen deposited within chamber 555 during collection procedures. Movement of shield 550, relative to chamber 555 of sealing plug 541 can include a third position, whereby aperture 575 would be locked in a closed position, preventing external access to chamber 555.

Container 540 does not have to be opened to gain access to collected specimen in internal cavity 543, nor does sealing plug 541 have to be punctured with a needle, or removed to obtain a small amount of specimen for visual analysis.

Chamber 555 can be coated with an anti-clotting agent, dye or the like to facilitate visual examination of the specimen.

FIG. 68 is a cross sectional side view of the collection container shown in FIG. 66 having chamber 543 being closed by removable sealing plug 541 with movable external shield 550 having aperture 575 closing chamber 555 of sealing plug 541 at section 576. External shield 550 is in a first position closing external access to chamber 555 of sealing plug 541.

As a needle moves through well 544 and punctures sealing plug 541, the hollow bore of the needle containing specimen enters chamber 555 depositing a small amount of specimen within the chamber. The needle then fully punctures the sealing plug 541 and specimen is collected in a normal fashion into chamber 543. When needle is removed from sealing plug 541, again a small amount of specimen is deposited within chamber 555.

FIG. 69 is a cross sectional side view of the collection container shown in FIG. 67 having chamber 543 being closed by removable sealing plug 541 with movable external shield 550 aperture 575 in a second position exposing chamber 555 allowing access to collected specimen deposited within chamber 555 during collection procedures.

Container 540 does not have to be opened, nor does sealing plug 541 have to be punctured with a needle, or removed to obtain a small amount of specimen for visual analysis.

A positive engagement means can position movable shield 550 in either a first closed position, or a second open position, reducing the probability of movable shield 550 inadvertently opening prematurely during the collection process.

A needle, with one sharpened tip for puncturing a sealing plug of a filled collection container, and the other end being blunted, can also be used to deposit a smear of blood on a slide. The needle can be attachable to a needle holder described throughout this application, or can be used individually to access a collected specimen in a collection container.

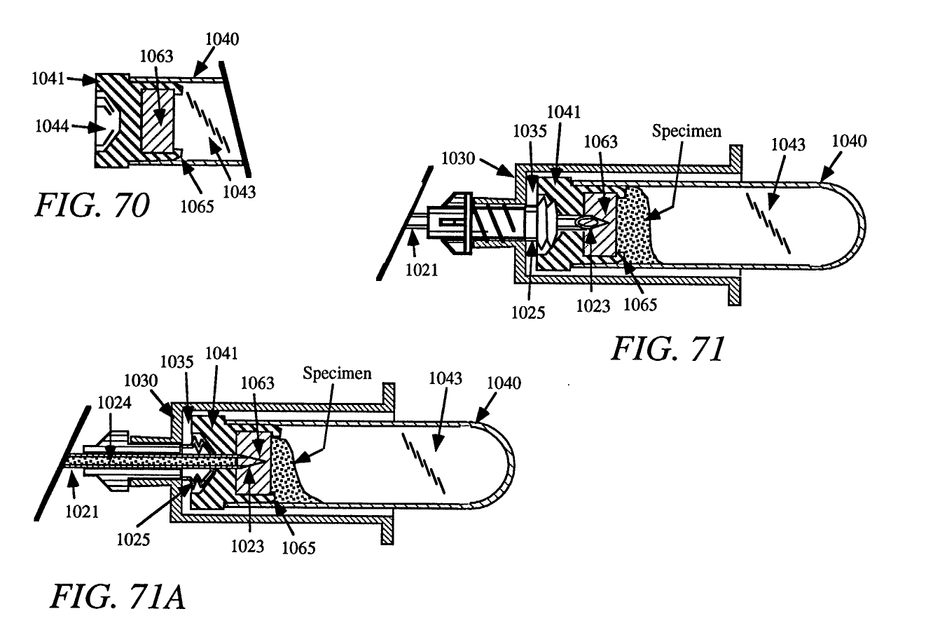

FIG. 70 is a cross sectional side view of the present invention having a container with sealing plug which includes a diffusing member. Sealing plug 1041 includes an external well 1044 and a porous member 1063 for diffusing a liquid entering internal chamber 1043 of container 1040 during the collection process. Sealing plug 1041 includes a chamfered bottom 1065 to facilitate insertion into container 1040.

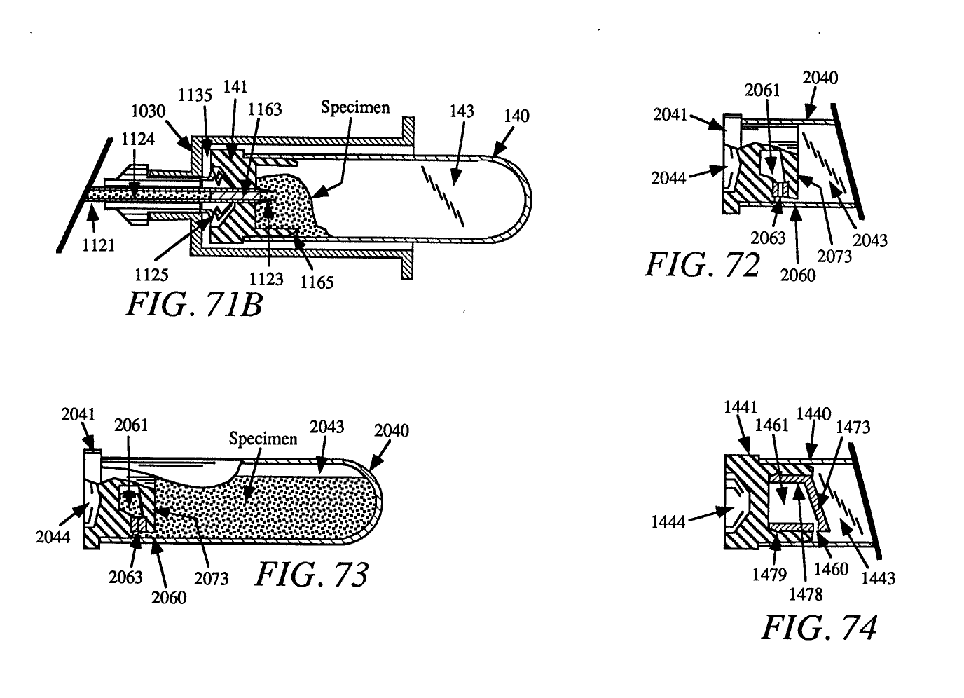

FIG. 71 is a cross sectional side view of the container and sealing plug shown in FIG. 70 showing a specimen being diffused during delivery into container 1040. Needle 1021 with pierceable resilient cover 1025 formed on the proximal end thereof is attached in needle holder 1030 and container 1040 is inserted in chamber 1035 of needle holder 1030, allowing proximal end of needle 1023 to puncture sealing plug 1041 and enter porous member 1063. Specimen exits needle 1021 and is diffused during the collection process. Sealing plug 1041 includes a chamfered bottom 1065 to facilitate insertion into container 1040.

FIG. 71A is a cross sectional side view of the container and sealing plug shown in FIG. 71 showing a specimen being diffused during delivery into container 1040. Specimen flows through needle lumen 1024 and into or through porous member 1063 before entering cavity 1043 of container 1040. Needle 1021 is attached in needle holder 1030 and container 1040 is inserted in chamber 1035 of needle holder 1030, allowing proximal end of needle 1023 to puncture sealing plug 1041 and enter porous member 1063. Specimen exits needle 1021 and is diffused during the collection process. Sealing plug 1041 includes a chamfered bottom 1065 to facilitate insertion into container 1040.

FIG. 71B is a cross sectional side view of a blood collection apparatus showing the specimen flowing through a porous regulating means 1163 within needle lumen 1124. Specimen flows through needle lumen 1124 and into or through porous member 1163 before entering cavity 143 of conventional container 140. Needle 1121 is attached in needle holder 1030 and container 140 is inserted in chamber 1135 of needle holder 1030, allowing proximal end of needle 1123 to puncture conventional sealing plug 141 and enter inner chamber 143. Specimen flows through needle 1121 and is diffused within needle lumen 1124 by porous member 1163 during the collection process. Sealing plug 141 includes a chamfered bottom 1065 to facilitate insertion into container 140. This fluid regulating needle allows a specimen to be collected using conventional blood collection containers, yet reduces both hemolysis and vein collapse probability.

FIG. 72 is a cross sectional side view of the present invention having a container with sealing plug which includes an occluding member. Sealing plug 2041 includes an external well 2044, an inner chamber 2061, a diverting section 2073 and an expandable, liquid-sensitive section 2063 having an open aperture which allows a liquid to pass through it, yet expands and closes within minutes after being exposed to a liquid.

FIG. 73 is a cross sectional side view of FIG. 72 showing a specimen contained within container 2040 and chamber 2061 of sealing plug 2041 no longer being in fluid communication with chamber 1043 of container 1040. Liquid-sensitive section 2063 is swollen and the aperture closed.

FIG. 74 is a cross sectional side view of the present invention having a container with an existing prior art sealing plug or sealing plug 1441 with an external well 1444 which includes a diverting component 1478 having a chamber 1461 created by inserting diverter 1478 into hollow end of sealing plug 141. Diverter 1478 having a closed end 1473, at least one open aperture 1460 and may include at least one projection 1479 for frictionally engaging inner wall of sealing plug 141. Diverter 1478 may include a chamfered closed end 1473 to allow specimen remaining in chamber 1461 to drain into chamber 143 of container 140 prior to, or during centrifuging or analysis. Projection 1479 may be segmented or circumferential to support wall section of sealing plug 141, creating an improved seal at the sealing plug/container surface interface, thus reducing vacuum leakage and increasing the shelf life of the vacuum tube.

Diverter end 1473 may include a large opening and be closed by a removable or dissolvable wall section which allows the specimen remaining in the intermediate chamber 1461 after collection to be added to the specimen in the collection container chamber 1443. Specimen would still flow through aperture 1460 during the collection process. A dissolvable material, such as those used as additives to facilitate analysis, maybe used and dissolve within minutes of being wetted by a specimen. The diverting end 1473 would be removed from the diverter by the centrifugal forces placed on the container during the centrifuge process. Thus any specimen remaining in the intermediate chamber 1461 would be added to the specimen in the collection container. This would allow a long needle to freely access the specimen, which may be separated into plasma and red cells during the centrifuge process, enabling a pure plasma or red cell specimen to be drawn from the container for analysis.

Diverter 1478 radially compresses sealing plug 1441 against collection container 1440 wall, improving seal and increasing shelf life of the container. The inner wall section of sealing plug 1441 may include an annular or segmented undercut or projection to correspondingly mate with a projection or undercut respectively, on diverter 1478. An undercut or recess is easily moldable into elastomeric materials like rubber or rubber mixed with plastic, or other compounds.

The cost of implementing this flow diverting technology is very low because existing tooling and components do not have to be modified. The assembly procedure would have to be modified and tools created for manufacturing the diverting component.

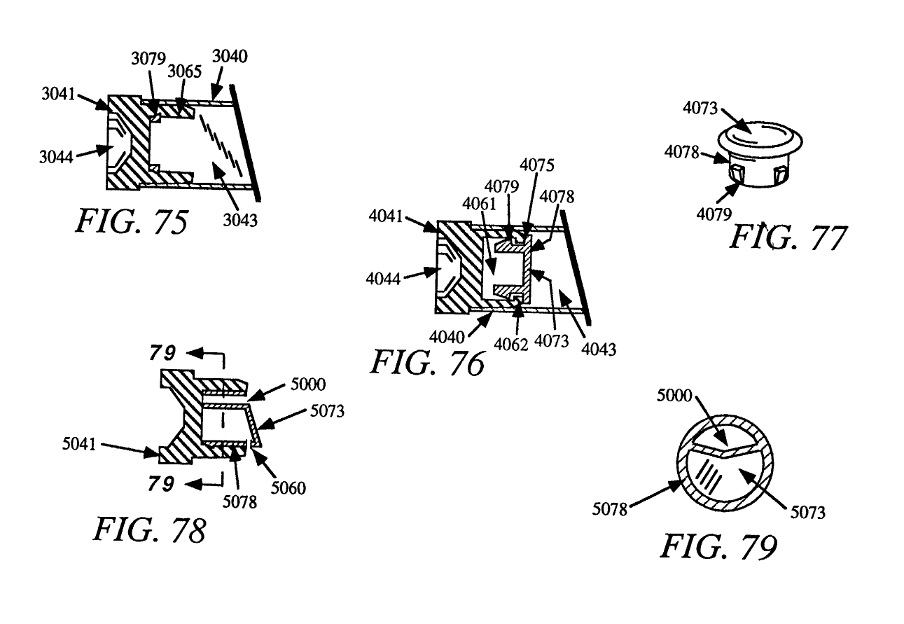

FIG. 75 is a cross sectional side view of the present invention having a container with an existing prior sealing plug with an annular member which frictionally engages the inner wall section of sealing plug. The sealing plug wall section 3065 adjacent to annular member 3079 compresses and supports the wall section 3065 when the sealing plug 141 is inserted in container 140. Annular member 3079 may be cylindrical in shape with open ends. Cylindrical configuration contacts a greater portion of sealing plug wall section, creating more surface area of contact with collection container wall. Annular member 3079 radially compresses sealing plug against collection container wall, improving the seal and increasing shelf life of the container.

The use of annular member 3079 provides an improved seal at the sealing plug/container surface interface, thus reducing “gray band” regions and vacuum leakage, thus increasing the shelf life of the vacuum tube.

The cost of implementing this improved sealing technology is very low because existing tooling and components do not have to be modified. The assembly procedure would have to be modified and tools created for manufacturing the annular component.

FIG. 76 is a cross sectional side view of the present invention having a container with a sealing plug with a pressure sensitive valve which is activated when a pressure difference exists on either side of the valve. Sealing plug 4041, includes an external well 4044 and projection or undercut 4062, which may have an annular configuration, is inserted in container 4040 creating cavity 4043. Valve 4078 is positioned in sealing plug 4041 and creates chamber 4061 which closes at interface 4075. When sealing plug 141 is punctured by a needle and a greater pressure is created in chamber 4061, valve 4078 opens and the area of higher pressure moves to the area of lower pressure contained in cavity 4043. Valve 4078 also has a diverting section 4073.

FIG. 77 is a full side view of the pressure sensitive diverter or valve 4078 which is inserted in a sealing plug.

FIG. 78 is a cross sectional side view of a sealing plug 5041 having a diverting component 5078 with an aperture 5060 for diverting or regulating a specimen flow, a diverting wall section 5073, and a channel or slot 5000 for accessing a collected specimen in a container sealed by sealing plug 5041. A long needle may be inserted through sealing plug 5041 to access the collected specimen with out contacting any specimen which may be residing in the chamber created by the coupling of sealing plug 5041 and diverter 5078.

FIG. 79 is a partial cut away view of the diverting component 5078 of FIG. 78. Diverting component 5078 having a channel or slot 5000 and chamber created by diverting wall section 5073.

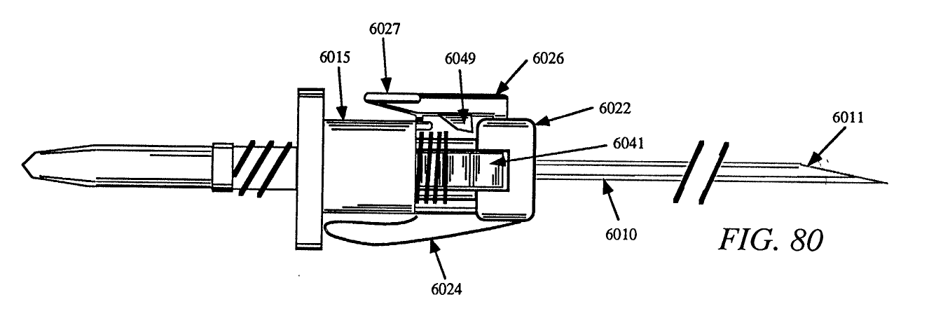

FIG. 80 is a full side view of a blood collection needle with a manually activated needle guard. Blood collection needle having a hub 6015, with a fixedly attached hollow bore needle 6010 with both the proximal and distal ends sharpened, distal end 6011 is shown here, with the proximal end covered by a puncturable boot or cover. A needle guard 6022 is releasably held adjacent to hub 6015 by a latching arm 6026 which includes a finger pad 6027 and a stop or projection 6049. Needle guard 6022 having a needle trap 6041 which rides on the needle and moves to a protecting position when needle guard 6022 is manually released by activating the finger pad 6027. Any longitudinal compressive force exerted by inserting needle 6010 into a patient does not activate needle guard.

1. A two-piece sealing plug for facilitating controlled regulation of a specimen flow to mitigate occurrence of hemolysis, the sealing plug comprising:
a pierceable section adapted to receive a needle with a lumen extending therein; and
a flow diverting section conformed to slide the needle therethrough and having a first and a second end, the first end being attachable to the pierceable section, the second end forming a plurality of alternate configurations;
wherein the plurality of alternate configurations facilitate the controlled regulation of the specimen flow extruding from the lumen to mitigate the occurrence of hemolysis.

2. The sealing plug of claim 1 wherein the first end is removably attachable to the pierceable section.

3. The sealing plug of claim 1 wherein the pierceable section is greater in size than the flow diverting section.

4. The sealing plug of claim 1 wherein the pierceable section and the flow diverting section are unitarily formed.

5. The sealing plug of claim 1 wherein the second end comprises a diverter and a recess jointly forming at least one channel, the at least one channel being configured to have a plurality of sizes to generate different rates of the specimen flowing therethrough.

6. The sealing plug of claim 5 wherein the sizes of the at least one channel is smaller than an area of the lumen to decrease the rate of the specimen flowing therethrough.

7. The sealing plug of claim 5 wherein the sizes of the at least one channel is equal to an area of the lumen to result in the specimen flowing freely therethrough.

8. The sealing plug of claim 5 wherein the sizes of the at least one channel is greater than an area of the lumen to result in the specimen flowing freely therethrough.

9. The sealing plug of claim 5 wherein the diverter guides the specimen flowing therethrough.

10. The sealing plug of claim 1 wherein the second end comprises a plurality of internal chambers for sliding the needle therethrough.

11. The sealing plug of claim 10 wherein the internal chambers form a plurality of uniformly sized apertures, the apertures being selectively sized to generate different rates of the specimen flowing therethrough.

12. The sealing plug of claim 11 wherein the size of the apertures is reduced to decrease the rate of the specimen flowing therethrough.

13. The sealing plug of claim 11 wherein the size of the apertures is augmented to increase the rate of the specimen flowing therethrough.

14. The sealing plug of claim 11 wherein the apertures are contoured to guide the specimen flowing therethrough.

15. The sealing plug of claim 1 wherein the second end has at least one contoured aperture to guide the specimen flowing therethrough.

16. The sealing plug of claim 15 wherein the at least one aperture is selectively sized to generate different rates of the specimen flowing therethrough.

17. A collection container with mitigated vacuum leakage to increase shelf life thereof, the collection container comprising:
a container body having an internal chamber with an open end;
a sealing plug having a flow diverting section with an inner wall and an outer wall, the flow diverting section being engageable to the internal chamber through the open end thereof; and
at least one annular member disposed along the inner wall to exert a compressive force therealong such that the outer wall extends against the internal chamber to mitigate the vacuum leakage to increase the shelf life thereof.

18. The collection container of claim 17 wherein the container body is fabricated from glass.

19. The collection container of claim 17 wherein the container body has an outer surface with a shatter resistant coating thereon.

20. The collection container of claim 19 wherein the shatter resistant coating is a polymeric material.

21. The collection container of claim 17 wherein the flow diverting section is removably engageable around the internal chamber through the open end thereof.

22. The collection container of claim 17 wherein the sealing plug has a needle-pierceable section adapted to receive a needle, the needle-pierceable section being in abutting contact with the open end when the flow diverting section engages the internal chamber.

23. The collection container of claim 22 further comprising a specimen, wherein the flow diverting section is conformed to slide the needle therethrough for ingress and egress of the specimen from the collection container.

24. The collection container of claim 23 wherein the needle comprises a manually activated needle guard to protect the needle when inserted into the collection container.

25. The collection container of claim 24 wherein a finger pad is mechanically coupled to the needle guard, the finger pad being operative to release the needle guard when activated.

26. The collection container of claim 17 further comprising a shield, the shield being connectable with the sealing plug to facilitate removal of the sealing plug from the collection container.

27. The collection container of claim 17 wherein the at least one annular member frictionally engages the inner wall of the sealing plug.

28. The collection container of claim 17 wherein the at least one annular member is cylindrical in shape to increase surface area of contact of the outer wall with the internal chamber.

29. The collection container of claim 17 wherein the at least one annular member radially compresses the inner wall to enhance force of contact between the outer wall and the internal chamber.
